(19) United States
(12) Patent Application Publication
(10) Pub. No.: US 2022/0000805 A1
(43) Pub. Date: Jan. 6, 2022

(54) COMBINATION OF M-OPIOID RECEPTOR (MOR) MODULATORS FOR PREVENTING AND TREATING PAIN, SUICIDALITY AND MENTAL DISORDERS

(71) Applicants: HADAS IT MEDICAL RESEARCH SERVICES & DEVELOPMENT LIMITED, Jerusalem (IL); YISSUM RESEARCH DEVELOPMENT COMPANY OF THE HEBREW UNIVERSITY OF JERUSALEM LTD., Jerusalem (IL)

(72) Inventors: Yoram YOVELL, Kibbutz Kiriat Anavim (IL); Shmuel BEN-SASSON, Jerusalem (IL)

(21) Appl. No.: 17/290,012

(22) PCT Filed: Oct. 29, 2019
(86) PCT No.: PCT/IL2019/051170

§ 371 Date: Apr. 29, 2021

Related U.S. Application Data
(60) Provisional application No. 62/752,036, filed on Oct. 29, 2018.
(60) Provisional application No. 62/811,560, filed on Feb. 28, 2019.

(51) Int. Cl. A61K 31/135; A61K 31/554; A61K 31/407; A61P 29/00

(52) U.S. Cl. CPC A61K 31/135; A61P 29/00; A61K 31/407; A61K 31/554

(57) ABSTRACT

The invention provides synergistic combinations of at least two modulators of MOR, as well as combined compositions, kits methods and uses thereof for treating mental and physical pain, suicidality and depression.

### FIELD OF THE INVENTION

The present invention relates to combined therapy of suicidality, physical pain, and mental disorders. More particularly, the invention relates to synergistic combinations of specific μ-opioid receptor (MOR or mu receptor) modulators, pharmaceutical compositions, uses, kits and methods thereof for treating and preventing acute suicidality, physical pain, mental pain and depression.

### BACKGROUND ART

References considered to be relevant as background to the presently disclosed subject matter are listed below:
[1] Yovell Y. et al., Am J Psychiatry 2015 (doi:10.1176/appi.ajp.2015.15040535);
[2] WO 2013/042054;
[3] WO 2013/088242;
[4] Ballard E D et al. J Psychiatr Res 2014; 58:161-166;
[5] Lapidus K A B et al. Biol Psychiatry 2014; 76:970-976;
[6] Domany Y, et al. The British Journal of Psychiatry 2018; 1-7. doi:10.1192/bjp.2018.196;
[7] Williams N R et al. Am J Psychiatry. 2018 Aug. 29:appiajp201818020138. doi: 10.1176/appi.ajp.2018.18020138];
[8] Gassaway M M, et al. Transl Psychiatry (2014) 4, e411;
[9] Courtet P et al. Journal of Psychiatric Research 2018; Volume 96, Pages 167-170;
[10] Kruegel et al. J Am Chem Soc. (2016) 138(21): 6754-6764.
[11] Burford N T et al. PNAS (2013) vol. 110; no. 26, p 10830-10835.

Acknowledgement of the above references herein is not to be inferred as meaning that these are in any way relevant to the patentability of the presently disclosed subject matter.

### BACKGROUND OF THE INVENTION

Suicide, with a worldwide annual mortality approaching one million, is anteceded by suicidal ideation, thoughts and wishes to kill oneself (Hawton K, et al. Lancet 2009; 373:1372-1381). Standard antidepressants relieve suicidal ideation, but this may take several weeks, and not all patients respond adequately (Barbui C, et al. CMAJ 2009; 180:291-297). Mental pain and physical pain are associated with suicidality, in addition to their association with depression and separation distress. Some studies have found mental pain to be the psychological variable most strongly associated with current suicidality, more so than depression and hopelessness (Troister T, et al. Pers Individ Dif 2010; 49:689-693). Opioids were widely used to treat depression from about 1850 to 1956. Because of their addictive potential and lethality in overdose, opioids were replaced by standard antidepressants once these became available. However, several studies since then have found them to be effective for treating depression (Tenore P L. J Addict Dis 2008; 27:49-65). Buprenorphine, which is utilized in high dosages for the treatment of opioid use disorder, has a complex pharmacological profile. It is a partial mu agonist and a potent kappa antagonist, and has several active metabolites (Butler S: et al. Scand J Pain 2013; 4:148-152). Its kappa antagonism may enhance its antidepressant action (Ehrich E, et al: Neuropsychopharmacology 2015; 40:1448-1455). It causes much less respiratory depression than other opioids and is therefore safer in overdose (Dahan A, Yassen A, et al. Br J Anaesth 2006; 96:627-632). The inventors have previously shown that time-limited short term use of low dosages of sublingual buprenorphine was associated with decreased suicidal ideation [1]. WO 2013/042054 [2], that is a previous publication of the inventors, discloses methods and compositions for treating acute suicidality by administration of buprenorphine at unit dosage forms comprising less than 0.2 mg, however further reduction of the amount required by synergistically combining several μ-opioid receptor (MOR) activators, is not suggested by this publication. WO 2013/088242 [3] relates compositions comprising combinations of buprenorphine and MOR antagonist (specific compound named SAMIDORPHAN (ALKS 33)) in a particular agonist/antagonist activity index of between about 0.7 to 2.2, for treating depressive disorders.

Ketamine, a glutamate receptor modulator, is effective as a quick-acting treatment for suicidal ideation and depression, but necessitates repeated administration under medical supervision [4-5]. Recently, it was reported that repeated oral ketamine produced amelioration of depressive symptoms in out-patients with treatment-resistant depression (TRD) [6]. Using ketamine and MOR antagonist, specifically, naltrexone, Schatzberg A F et al., showed that ketamine's acute antidepressant effect requires opioid system activation [7]. Thus, ketamine can be defined as μ-opioid receptor (MOR) modulator.

Tianeptine, an approved antidepressant, is a μ-opioid receptor (MOR) agonist that acts specifically on the opioidergic system [8]. Tianeptine was associated with lower risk of suicidal ideation worsening during the first weeks of treatment onset compared with other antidepressants [9].

Currently, there still remains a need to develop short-term pharmacological treatments that enhance the effect of therapeutic compounds suitable for independent outpatient use for treating suicidal ideation, major mental disorders and physical pain.

### SUMMARY OF THE INVENTION

In a first aspect, the invention relates to a combination of at least two μ-opioid receptor (MOR) modulators or a combined composition comprising a combination of said at least two μ-opioid receptor (MOR) modulators. In some embodiments, the combinations or compositions of the invention may optionally further comprise at least one pharmaceutically acceptable carrier, diluent, excipient and/or additive. In more specific embodiments, the combinations or combined compositions of the invention may comprise at least two of buprenorphine, ketamine and tianeptine, and/or any derivatives thereof or any pharmaceutically acceptable salts, esters, metabolites or prodrugs thereof.

A further aspect of the invention relates to synergistic combinations of MOR modulators, specifically, modulators that synergistically activate MOR. In more specific embodiments, the synergistic combinations of the invention may comprise at least two of buprenorphine, ketamine and tianeptine, and/or any derivatives thereof or any pharmaceutically acceptable salts, esters, metabolites or prodrugs thereof.

In yet another aspect, the invention provides a kit comprising at least two MOR modulators. In some optional embodiments, each MOR modulator of the kit of the invention, may be provided in a pharmaceutical dosage form. It should be noted that the kit may optionally further comprise container means for containing said dosage forms.

In yet some further aspects thereof, the invention provides methods for activating MOR in a cell. More specifically, the method of the invention may comprise the steps of contacting said cell with an effective amount of at least two MOR modulators, specifically, any of the activating modulators discussed by the invention, and combinations thereof, any combined compositions thereof or any kits comprising the at least two activating MOR modulators of the invention. In more specific embodiments, the method of the invention may comprise the step of contacting the cell with an effective amount of at least two of buprenorphine, ketamine and tianeptine. The invention further provide method for activating MOR in a subject in need by administering to said subject a synergistically effective amount of at least two of the MOR modulators of the invention, any combined compositions thereof or any kits comprising the same.

Another aspect of the invention relates to a method of treating, preventing, ameliorating, reducing or delaying the onset of at least one of suicidality, a mental disorder, mental pain and a physical pain in a subject in need thereof. In more specific embodiments, the methods of the invention may comprise the step of administering to the subject a therapeutically effective amount of at least two MOR modulators, any combinations thereof, any combined compositions thereof or any kit comprising the same, specifically, the combinations of the invention.

In yet another aspect, the invention provides a method of treating, preventing, ameliorating, reducing or delaying the onset of at least one of suicidality, a mental disorder, mental pain and a physical pain, in subjects that are already treated with at least one MOR modulator.

Another aspect of the invention pharmaceutical composition for use in a method of treating, preventing, ameliorating, reducing or delaying the onset of at least one of at least one of suicidality, a mental disorder, mental pain and a physical pain, in a subject in need thereof, said composition comprises as an active ingredient a therapeutically effective amount of a combination of at least two MOR modulators and optionally at last one pharmaceutically acceptable carrier.

In yet another aspect, the invention provides at least two MOR modulators, or any compositions comprising the same, for use in a method of treating, preventing, ameliorating, reducing or delaying the onset of at least one of suicidality, a mental disorder, mental pain and a physical pain in a subject in need thereof.

These and other aspects of the invention will become apparent by the hand of the following figures.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Buprenorphine/Ketamine Gi Agonistic Interactions with MOR Histogram showing percentage of cAMP inhibition with different concentrations of Buprenorphine and Ketamine combinations.

FIG. 2: Buprenorphine/Ketamine synergistic activation of MOR Histogram showing percentage of MOR activation with different concentrations of Buprenorphine and Ketamine metabolite (hydroxynorketamine=HNK) combinations.

FIG. 3: Buprenorphine/Tianeptine Gi Agonistic Interactions with MOR Histogram showing percentage of cCAMP inhibition with different concentrations of Buprenorphine and Tianeptine combination.

FIG. 4: Buprenorphine/Tianeptine synergistic activation of MOR Graph showing percentage of MOR activation with different concentrations of Buprenorphine and Tianeptine.

FIG. 5: Tianeptine/Ketamine synergistic activation of MOR Histogram showing percentage of MOR activation with different concentrations of Tianeptine and Ketamine metabolite (hydroxynorketamine=HNK) combinations.

FIG. 6: MOR Positive Control Dose-Response Curve Graph showing percentage of cAMP inhibition with different concentrations of Enkephalin.

### DETAILED DESCRIPTION OF THE INVENTION

The present invention, in some embodiments thereof, relates to combined therapy and more particularly, but not exclusively, to combined compositions, kits, uses and methods for the treatment of acute suicidality, mental disorders and physical pain. The principles and operation of the present invention may be better understood with reference to the figures and accompanying descriptions. Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

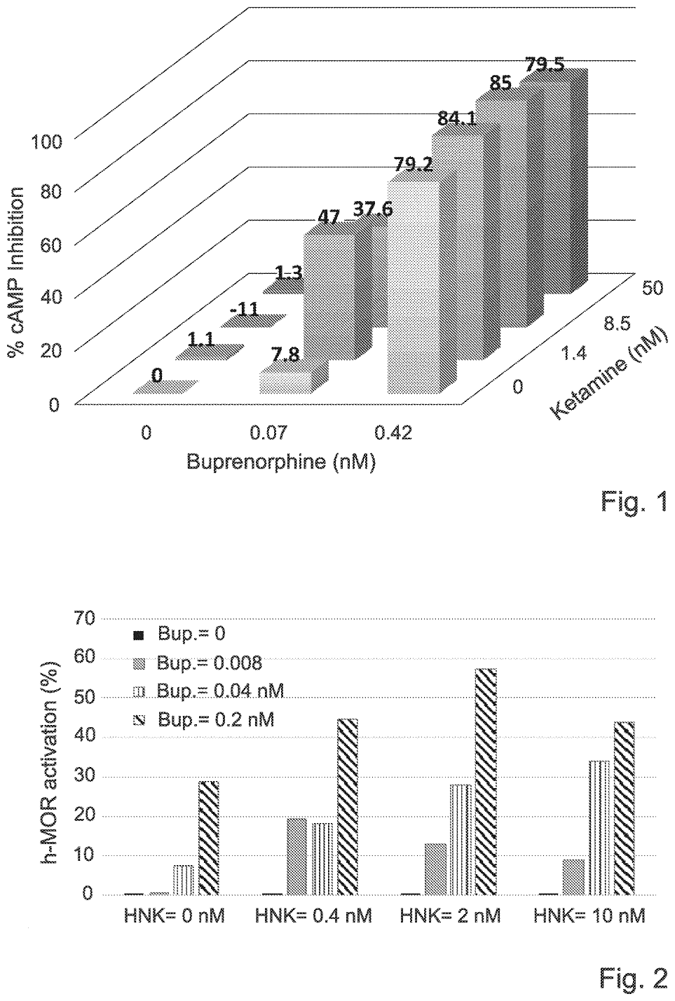

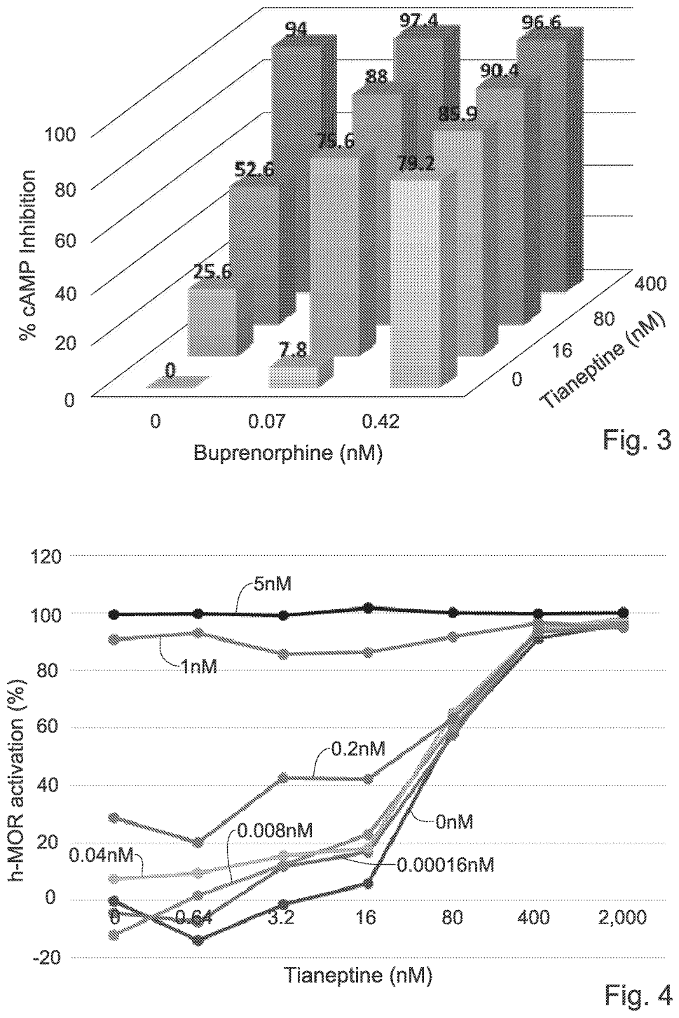

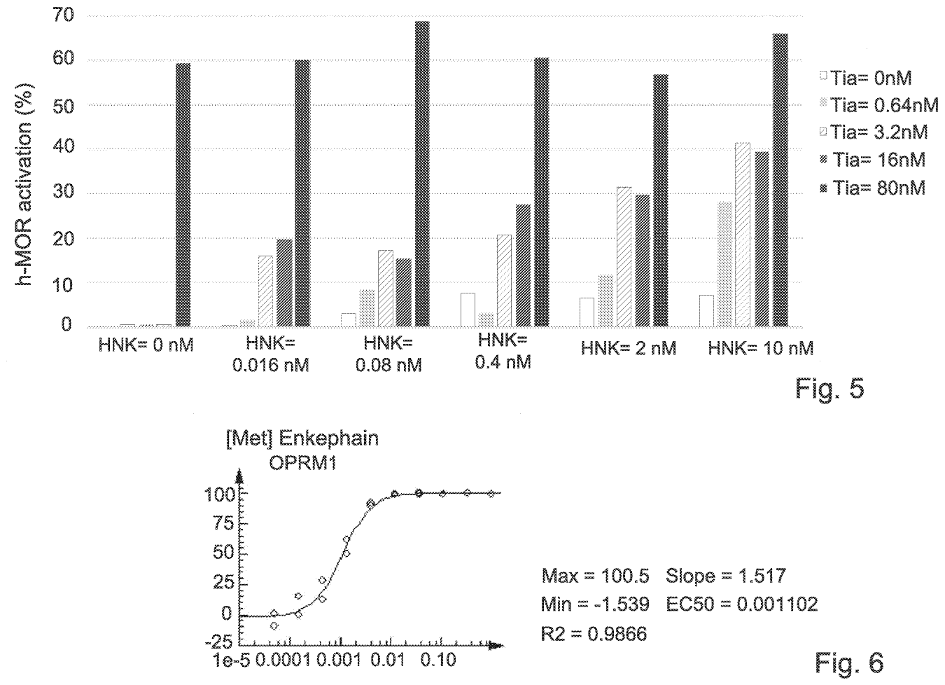

As discussed hereinabove, there is no current effective treatment against acute suicidality. In searching for an effective treatment of acute suicidality, the present inventors envisioned that acute suicidality can be treated with a synergistic combination of μ-opioid receptor (MOR) modulators, specifically, modulators that activate directly or indirectly the MOR, more specifically, buprenorphine, ketamine and tianeptine. While reducing the present invention to practice, the inventors uncovered surprising synergistic effects of ultra-low doses of MOR modulators, specifically, buprenorphine (less than 1 mg/day), ketamine (less than 10 mg/day) and tianeptine (less than 5 mg/day) on activation of MOR, and inhibition of cAMP production, which allow for the design of particularly effective treatment regimens with minimal adverse side effects (and hence enhanced patient compliance). As exemplified herein, the present inventors have demonstrated that combinations of buprenorphine, ketamine and tianeptine synergistically activate MOR. Referring now to the drawings and tables, FIG. 1 and Table 1 show that combinations of 0.07 nM buprenorphine with 1.4 nM ketamine results in synergistic activation of MOR. FIG. 3 and Table 3 further show that combinations of 0.07 nM buprenorphine with 16 nM tianeptine synergistically activate MOR. FIG. 5 and Table 5, show synergistic activation of MOR by combining ketamine and tianeptine. These results therefore establish the feasibility of combining MOR activating modulators for synergistically enhancing the effect of each of these MOR activators on variety of conditions, for example, suicidality, mental disorders, mental and physical pain.

Thus, in a first aspect, the invention relates to a combination of at least two μ-opioid receptor (MOR) modulators, specifically, a synergistic combination or a combined composition comprising the combination of the at least two MOR modulators. In some embodiments, the composition of the invention may optionally further comprise at least one pharmaceutically acceptable carrier, diluent, excipient and/or additive. In some embodiments, the combined composition of the invention may be a pharmaceutical composition.

Opioid receptors have been targeted for the treatment of pain and related disorders for thousands of years, and opioid drugs remain the most widely used analgesics in the clinic. Mu (μ), kappa (κ), and delta (δ) opioid receptors represent the originally classified receptor subtypes, with opioid receptor like-1 (ORL1) being the least characterized. All four receptors are 7-transmembrane spanning proteins that couple to inhibitory G-proteins. These receptors form homo- and hetereodimeric complexes, signal to kinase cascades, and scaffold a variety of proteins. The μ-opioid receptor (MOR), also known as OPRM1, LMOR, M-OR-1, MOP, MOR, MOR1, OPRM, opioid receptor mu 1, is an inhibitory G-protein coupled receptor that activates the Gi alpha subunit, inhibiting adenylate cyclase activity (cAMP), thereby lowering cAMP levels. The μ-opioid receptors exist mostly presynaptically in the periaqueductal gray region, and in the superficial dorsal horn of the spinal cord (specifically the substantia gelatinosa of Rolando). MOR have been located in additional regions that include the external plexiform layer of the olfactory bulb, the nucleus accumbens, in several layers of the cerebral cortex, in some of the nuclei of the amygdala, as well as the nucleus of the solitary tract. More specifically, following activation by an agonist, such as the endogenous μ-opioid peptide endorphin, or exogenous agonists like morphine and fentanyl, the Gα and Gβγ subunits dissociate from one another and subsequently act on various intracellular effector pathways. The classical and perhaps most important aspect of opioid receptor signal transduction relates to their ability to modulate calcium and potassium ion channels. Following Gαᵢ dissociation from Gβγ, the Ga protein subunit moves on to directly interact with the G-protein gated inward rectifying potassium channel, Kᵢᵣ3. Channel deactivation happens following GTP to guanosine diphosphate (GDP) hydrolysis and Gβγ removal from interaction with the channel. This process causes cellular hyperpolarization and inhibits tonic neural activity. In several reports, the inhibitory effects of opioids on neural excitability were shown to be mediated by interactions of opioid receptors with G protein-regulated inwardly rectifying potassium channel (Kᵢᵣ3). It should be noted that in some embodiments, the MOR as used by the invention encompasses any mu-type opioid receptor known isoform, for example, the Homo sapiens MOR-1 as denoted by NCBI Reference Sequence: NP_000905.3, isoform MOR-1O as denoted by NP_001008503.2, mu-type opioid receptor isoform MOR-1A, as denoted by NP_001008504.2, mu-type opioid receptor isoform MOR-1X, as denoted by NCBI Reference Sequence: NP_001008505.2 and mu-type opioid receptor isoform MOR-1i, as denoted by NCBI Reference Sequence: NP_001138751.1, as denoted by SEQ ID NOs. 1, 2, 3, 4, and 5, respectively. In yet some further embodiments, the mu-type opioid receptor isoform in accordance with the invention may be encoded by genes located at chromosome 6q25.2, encoding the nucleic acid sequences (mRNAs) as denoted by NM_000914.4, NM_001008503.2, NM_001008504.3, NM_001008505.2, NM_001145279.3, respectively.

The present invention provides combinations of MOR modulators. As used herein, the term “ρ-opioid receptor modulator” or “MOR modulator” relates to a component, compound or agent that acts directly (e.g., by binding) or indirectly on the receptor, and leads to modulation of its activation, for example, increasing or enhancing its activation, thereby enhancing down-stream signaling thereof, e.g., inhibition of cAMP production.

MOR activation liberates G protein αi/o and βγ subunits, which modulate several effectors, including voltage-activated Ca²⁺ channels (VACCs), G-protein activated inwardly rectifying K⁺ (GIRK) channels and adenylyl cyclase. In addition, MOR activation also recruits arrestins 2 and 3 (β-arrestin 1 and 2), which participate in G protein coupled receptor (GPCR) desensitization, endocytosis and signaling through various kinases such as ERK1/2, Akt, JNK and c-Src. According to the invention, modulation may either activate and increase activation of MOR, or alternatively, decrease and inhibit activation of MOR, specifically, any of these indications, as specified herein. The term “modulator” therefore includes inhibitors and activators. In accordance with the invention, activators are agents that induce, activate, stimulate, increase, facilitate, enhance activation, sensitize or up regulate the activation of MOR, e.g., agonists, or any indirect activator. In some embodiments, activation of MOR results in at least one of: modulate calcium or potassium channels, inhibition of adenylyl cyclase production, equipment of arrestins 2 and 3 (β-arrestin 1 and 2), GPCR desensitisation, endocytosis and signaling through ERK1/2, Akt, JNK and c-Src. In some embodiments, activation of MOR by the activators of the invention results in inhibition of cAMP production. Inhibitors are agents that inhibit, partially or totally block stimulation or activation, decrease, prevent, delay activation, inactivate, desensitize, or down regulate the MOR activation in accordance with the invention, e.g., antagonists, or any indirect inhibitor. In some embodiments, inhibition of MOR by the inhibitors of the invention results in enhancement of cAMP production. It should be understood that the combined MOR modulators provided by the invention modulate any activity mediated by MOR, for example, inhibition or activation of various effector pathways, cAMP levels and modulation of calcium or potassium channels. MOR modulators as used herein, include naturally occurring and synthetic ligands, antagonists, agonists, small chemical molecules and the like.

It should be further understood that the invention encompasses any MOR modulator, for example, full agonists/antagonists and partial agonists/antagonists. Full agonists/antagonists produce a full biological response whereas partial agonists/antagonists only produce a partial biological response even at saturating concentrations. In yet some further optional and non-limiting embodiments, the term MOR modulator further encompasses allosteric modulator. More specifically, Allosteric modulation is used to alter the activity of molecules, receptors or enzymes. Allosteric modulation occurs when an effector binds to an allosteric site (also known as a regulatory site) of an enzyme or a receptor and alters the enzyme/receptor activity. Allosteric modulators are designed to fit the allosteric site to cause a conformational change of the enzyme/receptor, in particular a change in the shape of the active site, which then causes a change in its activity. In contrast to typical drugs, modulators are not competitive inhibitors. They can be positive (activating) causing an increase of the enzyme/receptor activity or negative (inhibiting) causing a decrease of the receptor/enzyme activity. As indicated above, opioid receptors are members of the class A family of GPCRs. Allosteric ligands for a GPCR bind to a site on the receptor that is distinct from the site that binds the orthosteric (or endogenous) agonist. An allosteric modulator can exhibit a range of activities at the target protein. Positive allosteric modulators (PAMs) may have no, or very little, intrinsic efficacy but, when they bind to the receptor, enhance the binding affinity and/or efficacy of the orthosteric agonist. Negative allosteric modulators (NAMs) have no intrinsic agonist efficacy but, when they bind to the receptor, inhibit the binding affinity and/or efficacy of the orthosteric agonist. Silent allosteric modulators (SAMs), also known as neutral allosteric ligands, bind to the receptor but have no effect on orthosteric agonist affinity or efficacy. However, SAMs can act as competitive antagonists at the same allosteric site, blocking PAM or NAM activity. Finally, allosteric agonists can bind and produce direct agonist activation of the receptor even in the absence of orthosteric agonist. Thus, as indicated above, in some alternative and non-limiting embodiments, the MOR modulators of the invention may act, at least in part, as allosteric modulators. In yet some further alternative or additional embodiments, the MOR modulators of the invention may further modulate additional opioid receptors, and/or other classes of receptors or any combinations thereof. In some specific embodiments, the modulators of the invention and/or any derivatives thereof may modulate homo- and heterodimers of MOR, δ-opioid receptor (DOR), κ-opioid receptor (KOR), and/or other classes of receptors.

In some embodiments, the modulator of the invention may be any compound or agent that modulates MOR, with the proviso that such modulating compound display no antagonistic activity on MOR. Thus, in some specific embodiments, the MOR modulator used by the combinations, compositions, kits, methods and uses of the invention may be an activating modulator that may act as an agonist, and in some embodiments, as an allosteric agonist, or alternatively, may indirectly activate the MOR (e.g., by enhancing the stability, expression, binding affinity of an agonist, or alternatively, by blocking antagonist activity). It should be noted that the term “agonist”, as used herein, relates to a compound, agent or drug that binds to a receptor, e.g., the MOR, and activates, stimulates, increases, activates, facilitates, enhances activation, sensitizes or up regulates the activity of the receptor to produce a biological response. The term “antagonist” relates to a compound, agent or a drug that binds to a receptor and partially or totally blocks stimulation, decreases, prevents, delays activation, inactivates, desensitizes, down regulates the activity or dampens a biological response.

According to some embodiments, wherein indicated “increasing” or “enhancing” the MOR activity, as used herein in connection with the MOR modulators of the invention, it is meant that such increase or enhancement may be an increase or elevation of between about 5% to 100%, specifically, 10% to 100% of the MOR activity. The terms “increase”, “augmentation” and “enhancement” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the activity as compared to a suitable control, e.g., MOR activation in the absence of the modulators of the invention. It should be further noted that increase or elevation may be also an increase of about 2 to 10⁶ folds or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. Therefore, the term increase refers to an increase of about 2, 3, 4, 5, 6, 7, 8, 9, 10, 20, 30, 40, 50, 60, 70, 80, 90, 100, 200, 300, 400, 500, 600, 700, 800, 900, 1000 folds or more. In some embodiments, the at least two MOR modulators comprised within the synergistic combinations or combined compositions of the invention synergistically activate MOR.

As indicated herein, the modulators comprised within the combinations, combined compositions and kits of the invention, and used by the methods described herein after, may be activating modulators of MOR that display a synergistic activation of MOR. Synergy, as used herein refer to the interaction or cooperation of two or more substances, compounds, or any other agents, specifically, the MOR activating modulators of the invention, to produce a combined effect greater than the sum of their separate effects. In other words, the MOR modulators of the invention are those exhibiting a sum of their separate effects on MOR activation that is greater than the sum of their separate effects on MOR activation. It should be understood that the synergistic effect of the combinations of the invention can be reflected in different parameters, for example, reduction in the amount of each compound required for achieving the desired effect, extending the total duration of the effect achieved, and reducing the frequency of administrations required for achieving the desired effect over time.

More specifically, as shown by Zhao et al. (Zhao X, et al. (2012) J Clin Pharmacol.; 74(2):304-14) a single iv bolus dose of 0.5 mg/kg racemic ketamine resulted in HNK serum concentrations of about 30 nM for at least 24 hrs, long-lasting potentiating effect following administration once a day. Thus, in some embodiments, this synergistic effect enables the use of lower concentrations of the combined MOR modulators that still exhibit relatively long-lasting effect, thereby allowing reduction of the frequency of administration of the MOR modulators as well, required for achieving the desired effect.

In more specific embodiments, MOR modulators, and specifically, MOR activating modulators may comprise at least one of Buprenorphine, Ketamine, Tianeptine, Dezocine, morphine, propoxyphene, Oxycodone, Fentanyl, Heroin, Hydrocodone, Hydromorphone, Levorphanol, Meperidine, Methadone, Oxymorphone, Butorphanol, Pentazocine, Tramadol, Mitragynine and Codeine, any derivatives thereof, or any pharmaceutically acceptable salts, esters, metabolites or prodrugs thereof.

As indicated above, in some embodiments, the synergistic combinations and/or combined compositions of the invention, as well as the kits, uses and methods described herein after, may comprise the use of buprenorphine, and/or any derivatives thereof or any pharmaceutically acceptable salts, esters, metabolites or prodrugs thereof. Buprenorphine, as used herein is an opioid drug. It is as a non-selective, mixed agonist—antagonist opioid receptor modulator, acting as a partial agonist of the MOR, and display some antagonistic activity on kappa opioid receptor (KOR). Buprenorphine has the following chemical structure, as denoted by Formula I:

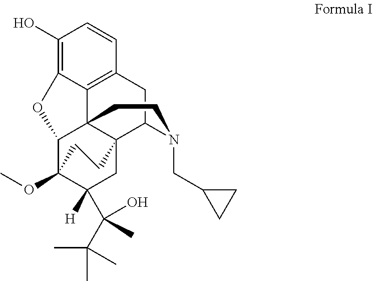

The systematic (IUPAC) name of buprenorphine is (2S)-2-[(−)-(5R,6R,7R,14S)-9a-cyclopropylmethyl-4,5-epoxy-6,14-ethano-3-hydroxy-6-methoxy morphinan-7-yl]-3,3-dimethylbutan-2-ol (C₂₉H₄₁N0₄; CAS number 52485-79-7). The molecular weight of the free base form of buprenorphine (depicted above) is 467.6 gram/mol. As shown hereinabove, buprenorphine has several chiral centers, and therefore various stereoisomers (e.g., diastereoisomers) of buprenorphine exist. It should be therefore appreciated that “Buprenorphine”, as used herein, encompasses any of the possible stereoisomers, including any mixture thereof. According to any of the embodiments described herein, the buprenorphine is substantially in the chiral form depicted hereinabove. Alternatively, the buprenorphine exists as a stereoisomer or enantiomer of the chiral form depicted hereinabove, or as a mixture of two or more chiral forms. It will be appreciated by one of skill in the art that the amount of the compound to be administered must be raised accordingly if an inactive chiral form is present. Still further, the term “buprenorphine” refer to buprenorphine in any pharmaceutically acceptable chemical and morphological form and physical state. These forms include without limitation buprenorphine in its free base form, protonated or partially protonated buprenorphine, buprenorphine salts, and in particular acid addition salts formed by addition of an inorganic or organic acid such as buprenorphine hydrochloride or buprenorphine sulphate, phosphate, tartrate, maleinate, oxalate, acetate, lactate, solvates, hydrates, clathrates, complexes and so on, as well as buprenorphine in the form of particles which may be micronized, crystalline and/or amorphous, and any mixtures of the aforementioned forms. The buprenorphine, where contained in a medium such as a solvent, may be dissolved or dispersed or in part dissolved and in part dispersed.

“Buprenorphine” further encompass pharmaceutically acceptable salts of buprenorphine and amorphous and crystalline states of buprenorphine or of a salt thereof, including any polymorph thereof. Buprenorphine, is sold under different brand names, for example, Temgesic (Buprenorphine hydrochloride, Sublingual tablet); Buprenex; Buprenorfina, Subutex (sublingual); Buprenorphinum; Butrans (skin patch), Belbuca (oral, buccal films), Sublocade, Suboxone contains a combination of buprenorphine and naloxone. Buprenorphine preparations are formulated for oral, nasal, buccal, injectable and transdermal administration.

It should be therefore understood that the term “buprenorphine” as used by the invention encompasses any form, brand, derivative, enantiomer, metabolite of the compound of Formula I, or any mixture thereof. In some specific embodiments, all buprenorphine produces disclosed herein, specifically, those that display the activating modulation on MOR, and more specifically, those that display synergistic activation of MOR when combined with other MOR modulators, may be applicable for each and every aspect of the invention, specifically, the combinations, combined compositions, kits, uses and methods described herein.

In yet some further specific embodiments, the synergistic combinations and/or combined compositions of the invention, as well as the kits, uses and methods described herein after, may comprise the use of ketamine, and/or any derivatives thereof or any pharmaceutically acceptable salts, esters, metabolites or prodrugs thereof. Ketamine is a medication that acts as a selective antagonist of the N-methyl D-aspartate (NMDA) receptor, an ionotropic glutamate receptor. It binds specifically to the dizocilpine (MK-801) site of the NMDA receptor, near the channel pore, and is an uncompetitive antagonist of NMDA-R. In addition, ketamine is an activating modulator of MOR, as stated above. Ketamine is used as an anesthetic, dissociative, and antidepressant drug. Ketamine has the following chemical structure, as denoted by Formula II:

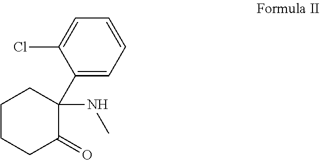 [SMILES: CNC1(c2ccccc2Cl)CCCCC1=O]

The systematic (IUPAC) name of Ketamine is (RS)-2-(2-Chlorophenyl)-2-(methylamino)cyclohexanone (C₁₃H₁₆ClNO; CAS number 6740-88-1). The molecular weight of Ketamine is 237.725 g/mol.

Ketamine is sold under different brand names, for example, Ketalar (ketamine hydrochloride injection), Calypsol, Ketamin, Ketamina, Ketamine, Ketaminol, Ketanest (Esketamine hydrochloride, containing), Ketaset, Tekam, and Vetalar among others, most contain Ketamine hydrochloride and are adapted for injection. Ketamine is also referred to as “K”, “KET”, “Special K”. Ketamine is a racemic mixture consisting of two enantiomers, R- and S-ketamine.

Esketamine, also known as (S)-ketamine or S(+)-ketamine and sold under the brand names Ketanest and Ketanest S. Still further, Hydroxynorketamine (HNK), or 6-hydroxynorketamine, is a metabolite of ketamine. It is formed by hydroxylation of the intermediate norketamine, another metabolite of ketamine. The major metabolite of ketamine is norketamine (80%). Norketamine is secondarily converted into 4-, 5-, and 6-hydroxynorketamines (15%). Ketamine is also transformed into hydroxyketamine (5%).

Specifically, in the predominant metabolic pathway, racemic ketamine [(R,S)-Ketamine] is initially metabolized to norketamine [(R,S)-norKetamine]. Subsequently, norketamine can be further metabolized to form the hydroxynorketamines. Hydroxylation of norketamine may result in formation of (2R,6R;2S,6S)-hydroxynorketamine or (2S,6R;2R,6S)-hydroxynorketamine or (2R,4R;2S,4S)-hydroxynorketamine or (2R,4S;2S,4R)-hydroxynorketamine or (2R,5S;2S,5R)-hydroxynorketamine or (2R,5R;2S,5S)-hydroxynorketamine (Zanos et al (2018) Pharmacol Rev. 70(3): 621-660).

In some embodiments, the metabolite of ketamine may be (2R,6R;2S,6S)-hydroxynorketamine or (2S,6R;2R,6S)-hydroxynorketamine or (2R,4R;2S,4S)-hydroxynorketamine or (2R,4S;2S,4R)-hydroxynorketamine or (2R,5S;2S,5R)-hydroxynorketamine or (2R,5R;2S,5S)-hydroxynorketamine. In some more specific embodiments, the metabolite of ketamine may be (2R,6R;2S,6S)-hydroxynorketamine.

It should be therefore understood that the term “ketamine” as used by the invention encompasses any form, brand, derivative, enantiomer, metabolite of the compound of Formula II or mixture thereof. In some specific embodiments, all ketamine produces disclosed herein, specifically, those that display the activating modulation on MOR, and more specifically, those that display synergistic activation of MOR when combined with other MOR modulators, may be applicable for each and every aspect of the invention, specifically, the combinations, combined compositions, kits, uses and methods described herein.

In some further embodiments, the synergistic combinations and/or combined compositions of the invention, as well as the kits, uses and methods described herein after, may comprise the use of tianeptine, and/or any derivatives thereof or any pharmaceutically acceptable salts, esters, metabolites or prodrugs thereof. Tianeptine is a tricyclic antidepressant (TCA), but has a very different drug profile than other TCAs. It acts as an atypical agonist of the μ-opioid receptor with clinically negligible effects on the δ- and κ-opioid receptors.

Tianeptine has the following chemical structure, as denoted by Formula III:

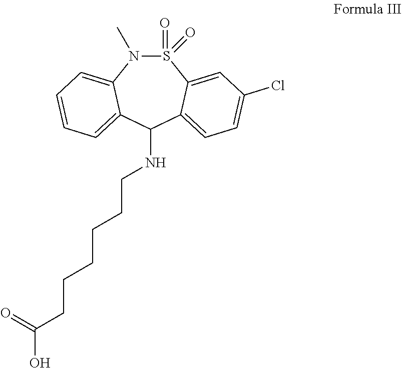 [SMILES: CN1c2ccccc2C(NCCCCCCC(=O)O)c2ccc(Cl)cc2S1(=O)=O]

The systematic (IUPAC) name of Tianeptine is 7-[(3-Chloro-6-methyl-5,5-dioxo-11H-benzo[c][2,1]benzothiazepin-11-yl)amino]heptanoic acid (C₂₁H₂₅ClN₂O₄S; CAS number 66981-73-5). The molecular weight of Tianeptine is 436.953 g/mol. As used herein, the term “tianeptine” refers to tianeptine compound, 7-[(3-chloro-6,11-dihydro-6-methyldibenzo[c,f] [1,2] thiazepin-11-yl)amino] heptanoic acid S,S-dioxide or a pharmaceutically acceptable salt, or an optical isomer, or a racemic mixture, or a prodrug, or a solvate, or a crystalline form thereof. The pharmaceutically acceptable salts of tianeptine include, for example, tianeptine hemisulfate, tianeptine sulfate, tianeptine hydrochloride, tianeptine phosphate, and tianeptine sodium salt.

Tianeptine is sold under the brand names Stablon, Coaxil, Salymbra, Tatinol and Tianeurax among others, most contain Tianeptine sodium salt hydrate or Tianeptine sulfate and is sold as a powder or capsules. It should be therefore understood that the term “tianeptine” as used by the invention encompasses any form, brand, derivative, enantiomer, metabolite of the compound of Formula III, or any mixture thereof. In some specific embodiments, all tianeptine produces disclosed herein, specifically, those that display the activating modulation on MOR, and more specifically, those that display synergistic activation of MOR when combined with other MOR modulators, may be applicable for each and every aspect of the invention, specifically, the combinations, combined compositions, kits, uses and methods described herein.

In yet some further embodiments, the synergistic combinations and/or combined compositions of the invention, as well as the kits, uses and methods described herein after, may comprise the use of dezocine, and/or any derivatives thereof or any pharmaceutically acceptable salts, esters, metabolites or prodrugs thereof. Dezocine, as used herein, is a marketed opioid analgesic of the benzomorphan group. It acts as a modulator of μ, δ, and κ-opioid receptors. Dezocine is a mixed agonist/antagonist of opioid receptors. It is a silent antagonist of the κ-opioid receptor, and in accordance, does not produce side effects such as dysphoria or hallucinations at any dose. Dezocine has the following chemical structure, as denoted by Formula IV:

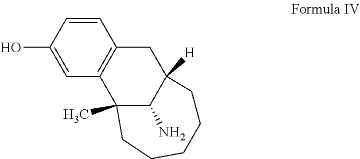 [SMILES: C[C@@]12CCCCC[C@@H](Cc3ccc(O)cc31)[C@@H]2N]

The systematic (IUPAC) name of Dezocine is (5R,11S,13R)-13-Amino-5-methyl-5,6,7,8,9,10,11,12-octahydro-5,11-methanobenzo[10]annulen-3-ol (C₁₆H₂₃NO; CAS Number 53648-55-8). The molecular weight of Dezocine is 245.36 g/mol. It should be understood that the term “Dezocine” as used by the invention encompasses any form, brand, derivative, enantiomer, metabolite of the compound of Formula IV, or any mixture thereof. In some specific embodiments, all Dezocine produces disclosed herein, specifically, those that display the activating modulation on MOR, and more specifically, those that display synergistic activation of MOR when combined with other MOR modulators, may be applicable for each and every aspect of the invention, specifically, the combined compositions, kits, and methods described herein.

Still further, in some embodiments, the synergistic combinations and/or combined compositions of the invention, as well as the kits, uses and methods described herein after, may comprise the use of morphine, and/or any derivatives thereof or any pharmaceutically acceptable salts, esters, metabolites or prodrugs thereof. Morphine, as used herein, is a pain medication of the opiate family which is found naturally in a number of plants and animals. It acts directly on the central nervous system (CNS) to decrease the feeling of pain. It interacts predominantly with the μ-δ-opioid (Mu-Delta) receptor heteromer.

Morphine has the following chemical structure, as denoted by Formula V:

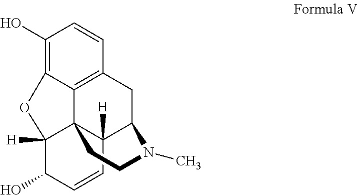

The systematic (IUPAC) name of Morphine is (4R,4aR,7S,7aR,12bS)-3-Methyl-2,3,4,4a,7,7a-hexahydro-1H-4,12-methanobenzofuro[3,2-e] isoquinoline-7,9-diol (C₁₇H₁₉NO₃; CAS number: 57-27-2). The molecular weight of morphine is 285.34 gram/mol. As shown hereinabove, Morphine has several chiral centers, and therefore various stereoisomers exist. “Morphine” therefore encompasses any of the possible stereoisomers, including any mixture thereof.

Morphine is sold under the following brand names MScontin, Oramorph, Sevredol, AVINza, Kadian, Morphabond, Oramorph, Roxanol, Duramorph and Infumorph among others. It should be understood that the term “morphine” as used by the invention encompasses any form, brand, derivative, enantiomer, metabolite of the compound of Formula V, or any mixture thereof. In some specific embodiments, all morphine produces disclosed herein, specifically, those that display the activating modulation on MOR, and more specifically, those that display synergistic activation of MOR when combined with other MOR modulators, may be applicable for each and every aspect of the invention, specifically, the combinations, combined compositions, kits, uses and methods described herein.

In some further embodiments, the synergistic combinations and/or combined compositions of the invention, as well as the kits, uses and methods described herein after, may comprise the use of dextropropoxyphene, and/or any derivatives thereof or any pharmaceutically acceptable salts, esters, metabolites or prodrugs thereof. Dextropropoxyphene or propoxyphene is an analgesic in the opioid category. Dextropropoxyphene is known under several synonyms, such as Alpha-d-4-dimethylamino-3-methyl-1,2-diphenyl-2-butanol propionate, [(2S,3R)-4-(Dimethylamino)-3-methyl-1,2-diphenylbutan-2-yl] propanoate, (+)-1,2-Diphenyl-2-propionoxy-3-methyl-4-di-methylaminobutane or Desoxypropiophen. Dextropropoxyphene acts as an mu-opioid receptor agonist. It also acts as a potent, noncompetitive a3(34 neuronal nicotinic acetylcholine receptor antagonist, as well as a weak serotonin reuptake inhibitor. Dextropropoxyphene has the following chemical structure as denoted by Formula VI:

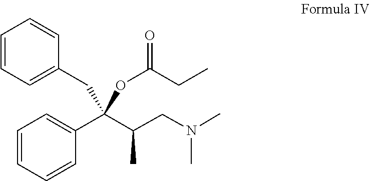 [SMILES: CCC(=O)O[C@](Cc1ccccc1)(c1ccccc1)[C@H](C)CN(C)C]

The systematic (IUPAC) name of Dextropropoxyphene is (1S,2R)-1-benzyl-3-(dimethylamino)-2-methyl-1-phenylpropyl propionate (C₂₂H₂₉NO₂; CAS number: 469-62-5). The molecular weight of Dextropropoxyphene is 339.471 gram/mol. Dextropropoxyphene is sold under the following brand names is Darvon, Darvocet-N, Di-Gesic, Dacoton and Saludex among others. It is to be understood that the term “Dextropropoxyphene” as used by the invention encompasses any form, brand, derivative, enantiomer, metabolite of the compound of Formula VI, or any mixture thereof. In some specific embodiments, all Dextropropoxyphene produces disclosed herein, specifically, those that display the activating modulation on MOR, and more specifically, those that display synergistic activation of MOR when combined with other MOR modulators, may be applicable for each and every aspect of the invention, specifically, the combinations, combined compositions, kits, uses and methods described herein.

In further embodiments, the synergistic combinations and/or combined compositions of the invention, as well as the kits, uses and methods described herein after, may comprise the use of Oxycodone, and/or any derivatives thereof or any pharmaceutically acceptable salts, esters, metabolites or prodrugs thereof. Oxycodone is an opioid medication which is used for the relief of moderate to severe pain. Oxycodone is a highly selective full agonist of the μ-opioid receptor (MOR). Oxycodone has low affinity for the δ-opioid receptor (DOR) and the κ-opioid receptor (KOR), where it is an agonist similarly. Oxycodone has the following chemical structure as denoted by Formula VII:

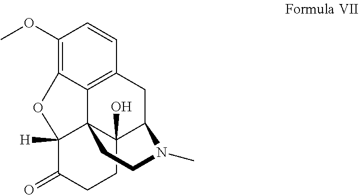

The systematic (IUPAC) name of Oxycodone is (5R,9R,13S,14S)-4,5α-Epoxy-14-hydroxy-3-methoxy-17-methylmorphinan-6-one (C₁₈H₂₁NO₄; CAS number: 76-42-6). The molecular weight of Oxycodone is 315.364 gram/mol. Oxycodone is sold under brand names such as Percocet, OxyContin, Roxicodone, Xtampza ER and OxyIR among others. Thus, the term “Oxycodone” as used by the invention encompasses any form, brand, derivative, enantiomer, metabolite of the compound of Formula VII, or any mixture thereof. In some specific embodiments, all Oxycodone produces disclosed herein, specifically, those that display the activating modulation on MOR, and more specifically, those that display synergistic activation of MOR when combined with other MOR modulators, may be applicable for each and every aspect of the invention, specifically, the combinations, combined compositions, kits, uses and methods described herein.

Still further, in some embodiments, the synergistic combinations and/or combined compositions of the invention, as well as the kits, uses and methods described herein after, may comprise the use of Mitragynine, and/or any derivatives thereof or any pharmaceutically acceptable salts, esters, metabolites or prodrugs thereof. Mitragynine is an indole-based opioid-receptor agonist and the most abundant active alkaloid in the plant Mitragyna speciose. Mitragynine itself acts primarily via μ-opioid receptors, though its oxidation product mitragynine pseudoindoxyl acts as a selective μ-opioid agonist with less affinity for δ or κ receptors. Another alkaloid with a major contribution to the μ-opioid activity of the kratom plant is the related compound 7-hydroxymitragynine, which, while present in the plant in much smaller quantities than mitragynine, is a much more potent μ-opioid partial agonist.

Mitragynine has the following chemical structure, as denoted by Formula VIII:

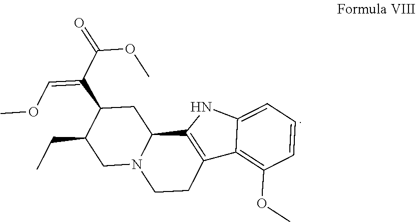 [SMILES: CC[C@@H]1CN2CCc3c([nH]c4cccc(OC)c34)[C@@H]2C[C@@H]1/C(=C\OC)C(=O)OC]

The systematic (IUPAC) name of Mitragynine is Methyl (2E)-2-[(2S,4S,5S)-5-ethyl-12-methoxy-7,17-diazatetracyclo[8.7.0.02,7.011,16]heptadeca-1(10),11(16),12,14-tetraen-4-yl]-3-methoxyprop-2-enoate (C₂₃H₃₀N₂O₄; CAS number: 4098-40-2). The molecular weight of Mitragynine is 398.4953 g/mol. Derivatives of Mitragynine comprise potent opioid agonists, notably some C10-halogen and 2,3-ethylene glycol bridged derivatives of 7-hydroxymitragynine. The most potent one is the 10-fluoro ethylene glycol adduct of 7-hydroxymitragynine with a potency 4-fold higher than 7-hydroxymitragynine. Other modification led to 2,3-dihydro derivatives such as MGM-15, prepared by reduction of 7-hydroxymitragynine with sodium borohydride. The anti-nociceptive effect of MGM-15 is between 15 and 50 times more potent than that of morphine after subcutaneous and oral administration.

In yet some further embodiments the combined compositions of the invention, as well as the kits, uses and methods described herein after, may comprise the use of any one of Fentanyl, Heroin, Hydrocodone, Hydromorphone, Levorphanol, Meperidine, Methadone, Oxymorphone, Butorphanol, Mitragynine, Pentazocine, Tramadol and Codeine.

Other MOR modulators that may be used in the combined compositions, kits, and methods of the invention may include at least one of alfentanil, allylprodine, alphaprodine, anileridine, benzylmorphine, bezitramide, brifentanil, carfentanil, clonitazene, dextromoramide, desomorphine, diampromide, diamorphone, dihydrocodeine, dihydromorphine, dimenoxadol, dimepheptanol, dimethyl-thiambutene, dioxaphetyl butyrate, dipipanone, eptazocine, ethoheptazine, ethylmethylthiambutene, ethylmorphine, etonitazene, etorphine, hydrocodeine, hydroxypethidine, isomethadone, ketobemidone, levallorphan, levomethadone, levophenacylmorphan, lofentanil, pethidine, metazocine, metopon, 4-methoxymethylfentanyl, 3-methylfentanil, mirfentanil, 6-monoacetylmorphine, morphine, morphine-6-glucuronide, ohmefentanyl, oxycodone, propoxyphene, propiram, propoxyphene, racemorphan, sufentanil, tapentadol and tilidine.

In yet some further embodiments the combined compositions of the invention, as well as the kits, uses and methods described herein after, may comprise the use of the compounds described in Burford et al. [11] that were identified as μ-opioid receptor PAMs and are denoted as BMS-986121 and BMS-986122 or any derivatives thereof.

As indicated above, any of the MOR modulator of the invention, specifically, those that display synergistic activation of MOR when combined with other MOR modulators, and more specifically, any of the activating modulators described herein, as well as any salts, esters, metabolite, enantiomers, isomorph and prodrug thereof, may be applicable in each and every aspect of the invention, specifically, any of the combinations, compositions, kits, methods and uses of the invention.

As used herein, the term “salts” and/or “pharmaceutically acceptable salts” refer to derivatives of the disclosed compounds wherein the parent compound is modified by making acid or base salts thereof, and/or quaternary salts of basic nitrogen-containing groups. Acid addition salts can be prepared by reacting compounds, in free base form, with inorganic or organic acids. Examples of inorganic acids include, but are not limited to, hydrochloric, hydrobromic, hydroiodic, nitric, carbonic, sulfuric, and phosphoric acid. Examples of organic acids include, but are not limited to, lauric, acetic, trifluoroacetic, formic, propionic, succinic, glycolic, lactic, malic, tartaric, citric, ascorbic, maleic, fumaric, pyruvic, aspartic, glutamic, stearic, salicylic, mandelic, methanesulfonic, benzenesulfonic, isoethonic, sulfanilic, adipic, butyric, oxalic, and pivalic. Base addition salt can be prepared by reacting compounds, in free acid form, with inorganic or organic bases. Examples of inorganic base addition salts include alkali metal salts, alkaline earth metal salts, and other physiologically acceptable metal salts, for example, aluminium, calcium, lithium, magnesium, potassium, sodium, or zinc salts. Examples of organic base addition salts include amine salts, for example, salts of trimethylamine, diethylamine, ethanolamine, diethanolamine, and ethylenediamine. Quaternary salts of basic nitrogen-containing groups in the compounds may be may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. Such conventional non-toxic salts may include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The term “esters” as used herein means chemical compounds derived from an acid (organic or inorganic) in which at least one —OH (hydroxyl) group is replaced by an —O-alkyl (alkoxy) group. Usually, organic esters are derived from a carboxylic acid and an alcohol. They may contain from 2-40 or more C atoms. Non-limiting examples of organic esters that may be used for the present invention include ethyl formate, methyl acetate, ethyl acetate, isopropyl acetate, ethyl malonate, and mixtures thereof. Inorganic esters are derived from an inorganic acid and an alcohol. The inorganic acids may be selected from e.g. phosphoric acid, sulfuric acid, nitric acid and boric acid. The term “metabolites” refers to all molecules derived from any of the compounds according to the present invention in a mammal cell or organism. The term relates to molecules which differ from any molecule which is present in any such cell or organism under physiological conditions. It pertains to the compounds formed by any metabolic process in-vivo, upon administration of the compound.

The term “prodrug”, as used herein refers to a compound that, after administration, is metabolized (i.e., converted within the body) into a pharmacologically active drug. Inactive prodrugs are pharmacologically inactive medications that are metabolized into an active form within the body. In some embodiments, the synergistic combinations and/or combined compositions of the invention, as well as the kits, uses and methods described herein after, may combine at least two MOR modulators, at least three, at least four, at least five, at list six, at least seven, at least eight, at least nine, at least ten or even more MOR modulators, specifically, activating modulators of MOR, and more specifically, any of the MOR modulators disclosed by the invention.

In some specific embodiments, the synergistic combinations and/or combined compositions of the invention may combine, and therefore comprise two MOR modulators, specifically, two activating modulators of MOR.

The present invention thus also contemplates derivatives of any compound of the present invention, specifically, any of the MOR modulators described herein. The term “derivative” as used herein refers to a chemically modified compound derived from a parent compound of the invention (e.g., Buprenorphine, Tianeptine and Ketamine etc.) that differs from the parent compound by one or more elements, substituents and/or functional groups such that the derivative has the same or similar biological properties/activities as the parent compound, as defined herein, specifically, in modulation of MOR.

More specifically, as described herein, compounds disclosed herein, specifically, the MOR modulators of the invention, more specifically, the compounds of any one of Formulas I, II, III, IV, V, VI, VII, VIII, may optionally be substituted with one or more substituents, thereby providing the derivatives of the invention.

In general, the term “substituted” means that the specified group or moiety bears one or more suitable substituents, in some embodiments the term refers to the replacement of one or more hydrogen radicals in a given structure or group with the radical of a specified substituent. Unless otherwise indicated, a substituent may have a substituent at each substitutable and reasonable position of the group. When more than one position in a given structure can be substituted with more than one substituent selected from a specified group, the substituent may be either the same or different at each position. The substituents disclosed herein including, but not limited to D, F, Cl, Br, I, —N₃, —CN, —NO₂, —OH, —SH, —NH₂, alkyl, alkenyl, alkynyl, haloalkyl, alkoxy, alkylthio, aminoalkyl, cycloalkyl, heterocyclyl, aryl, heteroaryl, and the like.

It should be noted that the term “optionally substituted” and “unsubstituted or substituted” can be used interchangeably herein, which means that the structure is unsubstituted or substituted by one or more substituents disclosed herein, wherein the substitution occurs at any valence allowable and reasonable site of structures or groups provided herein.

As used herein, the expression “one or more substituents” denotes one to maximum possible number of substitution (s) that can occur at any valency-allowed position on the system. In a certain embodiment, one or more substituent means 1, 2, 3, 4, or 5 substituents. In another embodiment, one or more substituent means 1, 2, or 3 substituents.

Any atom that is represented herein with an unsatisfied valence is assumed to have the sufficient number of hydrogen atoms to satisfy the atom's valence.

More specifically, as used herein, “alkyl” refers to a saturated, straight- or branched-chain hydrocarbon group having from 1 to 12 carbon atoms. Representative alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2, 2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2, 2-dimethyl-1-butyl, 3, 3-dimethyl-1-butyl, 2-ethyl-1-butyl, butyl, isobutyl, t-butyl, n-pentyl, isopentyl, neopentyl, n-hexyl, and the like, and longer alkyl groups, such as heptyl, octyl, and the like.

At various places in the present specification, substituents of compounds disclosed herein are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

Still further, the term “alkenyl” refers to a linear or branched-chain monovalent hydrocarbon radical containing 2 to 12 carbon atoms and at least one carbon-carbon, double bond, and includes radicals having “cis” and “trans” orientations, or alternatively, “E” and “Z” orientations. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃). The alkenyl radical may be optionally substituted with one or more substituents described herein.

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds.

Examples include, but are not limited to, —C≡CH, —C≡CH₃, —CH₂C≡CH₃, and —C≡CH₂CH(CH₂CH₃)₂.

The term “halogen” or “halo” are used interchangeably in this invention, and refers to Fluoro (F), Chloro (Cl), Bromo (Br), or Iodo (I).

The term “alkoxy” refers to an alkyl group, as previously defined, attached to the parent molecular moiety via an oxygen atom. Unless otherwise specified, the alkoxy group contains 1-12 carbon atoms. In one embodiment, the alkoxy group contains 1-6 carbon atoms. In other embodiment, the alkoxy group contains 1˜4 carbon atoms. In still other embodiment, the alkoxy group contains 1-3 carbon atoms. The alkoxy group may be optionally substituted with one or more substituents disclosed herein. As used herein, “alkoxyalkyl” means—(alkylenyl) —O— (alkyl), wherein each “alkyl” is independently an alkyl group defined above.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6-14, from 6 to 12, or from 6-10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “haloalkyl” refers to an alkyl group substituted with one or more halogen atoms, wherein the alkyl group is as defined herein. Some non-limiting examples of such groups include, but are not limited to —CF₃, —CF₂CF₃, —CH₂CF₂CHF₂, and the like. In one embodiment, “haloalkyl” refers to a lower C₁₋₄ haloalkyl, wherein the “C₁₋₄ haloalkyl” includes fluorine-substituted C1-4 haloalkyl, chlorine-substituted C₁₋₄ haloalkyl, bromine-substituted C₁₋₄ haloalkyl, iodine-substituted C₁₋₄ haloalkyl, and the like. Specifically, fluorine-substituted C₁₋₄ haloalkyl includes —CH₂F, —CHF₂, —CF₃, —CH₂Cl, —CHCl₂, —CCl₃, —CH₂Br, —CHBr₂, —CBr₃, —CH₂CH₂F, —CH₂CHF₂, —CH₂CF₃, —CF₂CH₂F, —CF₂CHF₂, —CF₂CF₃, —CHFCF₃, —CHFCHF₂, —CHFCH₂F, —CH₂CH₂CF₃, —CH₂CF₂CHF₂ and the like. The haloalkyl is optionally substituted with one or more substituents described herein.

The term “aminoalkyl” refers to an alkyl group substituted with one or more amino groups, wherein the alkyl group is as defined herein, and the amino group is optionally substituted.

The term “hydroxy-substituted alkyl” or “hydroxyalkyl” refers to an alkyl group substituted with one or more hydroxy groups, wherein the alkyl group is as defined herein. Some non-limiting examples of such group include, but are not limited to hydroxymethyl, hydroxyethyl, 1, 2-dihydroxyethyl, and the like.

The term “deuterium” as used herein means a stable isotope of hydrogen having one proton and one neutron.

The terms “carbocyclyl” and “carbocycle” as used interchangeably herein, refer to a monovalent or multivalent ring having 3 to 12 carbon atoms as a monocyclic, bicyclic or tricyclic ring system, which is saturated or contains one or more degrees of unsaturation, but an aromatic ring cannot exist in the carbocyclyl group.

The term “hydroxy” means an —OH group.

The terms “heterocyclyl” and “heterocycle” as used interchangeably herein refer to a monovalent or multivalent monocyclic, bicyclic or tricyclic ring containing 3-12 carbon atoms, wherein each one or more atoms in the ring is independently replaced with heteroatom, the heteroatom is as defined herein, and the ring may be saturated or contains one or more degrees of unsaturations, but an aromatic ring cannot exist in the aromatic ring.

The term “cycloalkyl” refers to a monovalent or multivalent saturated ring having 3 to 12 ring carbon atoms as a monocyclic, bicyclic, or tricyclic ring system.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl. Still further, it should be understood that any formula given herein is intended to represent compounds having structures depicted by the structural formula as well as certain variations or forms. For example, compounds of any one of formula I, II, III, IV, V, VI, VII, VIII, given herein may have asymmetric or chiral centers and therefore exist in different stereoisomeric forms. All stereoisomers, including optical isomers, enantiomers, and diastereomers, of the compounds of the general formula, and mixtures thereof, are considered to fall within the scope of the formula. Furthermore, certain structures may exist as geometric isomers (i.e., cis and trans isomers), as tautomers, or as atropisomers. All such isomeric forms, and mixtures thereof, are contemplated herein as part of the present invention. Thus, any formula given herein is intended to represent a racemate, one or more enantiomeric forms, one or more diastereomeric forms, one or more tautomeric or atropisomeric forms, and mixtures thereof.

“Stereoisomer” refers to compounds which have identical chemical constitution, but differ with regard to the arrangement of the atoms or groups in space. Stereoisomers include enantiomer, diastereomers, conformer (rotamer), geometric (cis/trans) isomer, atropisomer etc.

“Chiral” refers to molecules which have the property of non-superimposability of the mirror image partner, while the term “achiral” refers to molecules which are superimposable on their mirror image partner.

“Enantiomers” refers to two stereoisomers of a compound which are non-superimposable mirror images of one another.

“Diastereomer” refers to a stereoisomer with two or more centers of chirality and whose molecules are not mirror images of one another. Diastereomers have different physical properties, e.g., melting points, boiling points, spectral properties or biological activities. A mixture of diastereomers may be separated under high resolution analytical procedures such as electrophoresis and chromatography such as HPLC.

Thus, in some specific embodiments, the synergistic combinations and/or combined composition of the invention may comprise Buprenorphine and Ketamine, any derivatives thereof or any pharmaceutically acceptable salts, esters, metabolites or prodrugs thereof.

In yet some further embodiments, the synergistic combinations and/or combined compositions of the invention may comprise Buprenorphine and Tianeptine, any derivatives thereof or any pharmaceutically acceptable salts, esters, metabolites or prodrugs thereof.

In some further embodiments, the synergistic combinations and/or combined compositions of the invention may comprise ketamine and tianeptine, any derivatives thereof or any pharmaceutically acceptable salts, esters, metabolites or prodrugs thereof.

Still further, in some specific alternative embodiments, the synergistic combinations and/or combined compositions of the invention may combine, and therefore comprise three MOR modulators, specifically, three activating modulators of MOR.

Still further, in some embodiments, at least two of Buprenorphine, Ketamine, Tianeptine, may be further combined with at least one of Dezocine, morphine, propoxyphene, Oxycodone, Mitragynine, Fentanyl, Heroin, Hydrocodone, Hydromorphone, Levorphanol, Meperidine, Methadone, Oxymorphone, Butorphanol, Pentazocine, Tramadol, Mitragynine and Codeine any derivatives thereof, or any pharmaceutically acceptable salts, esters, metabolites or prodrugs thereof.

Thus, in some alternative embodiments, the synergistic combinations and/or combined compositions of the invention may comprise buprenorphine, ketamine and tianeptine, any derivatives thereof or any pharmaceutically acceptable salts, esters, metabolite or prodrugs thereof. In some embodiments, the combined compositions of the invention may further comprise at least one additional therapeutic agent.

As shown by the Examples, application of different combinations of MOR modulators, specifically, activating modulators such as buprenorphine, ketamine and tianeptine, using a cellular model, resulted in extensive and significant synergistic activation of MOR. The synergistic effect has been specifically enhanced in certain ranges of each MOR modulator. More specifically, as shown by the results, combination of 0.07 nM buprenorphine with 1.4 nM ketamine active metabolite (hydroxynorketamine=HNK) (see FIG. 1), as well as combination of 0.07 nM buprenorphine with 16 nM tianeptine (see FIG. 3), synergistically activates MOR. In addition, the results show that even lower concentrations of buprenorphine in combination with ketamine active metabolite (hydroxynorketamine=HNK), or tianeptine exhibit synergistic activation of MOR. Specifically, combination of 0.008 nM buprenorphine with 0.4 nM hydroxynorketamine (=HNK) (see FIG. 2) and combination of 0.008 nM buprenorphine with 3.2 nM tianeptine (see FIG. 4). Furthermore, in Example 3, very low concentrations of Ketamine active metabolite-hydroxynorketamine=HNK) and Tianeptine exhibit synergistic activation of MOR, specifically, concentration of 0.4 nM hydroxynorketamine (=HNK) with 3.2 nM Tianeptine (see FIG. 5). Thus, in some embodiments, buprenorphine, and/or any derivatives thereof or any pharmaceutically acceptable salts, esters, metabolites or prodrugs thereof in the combinations of the invention may be presented in an amount of about 0.001 or less to about 0.2 nM or more, more specifically, 0.001 nM, 0.002 nM, 0.003 nM, 0.004 nM, 0.005 nM, 0.006 nM, 0.007 nM, 0.008 nM, 0.009 nM, 0.01 nM, 0.02 nM, 0.03 nM, 0.04 nM, 0.05 nM, 0.06 nM, 0.07 nM, 0.08 nM, 0.09 nM, 0.1 nM, 0.2 nM or more. In more specific embodiments, 0.008 nM to about 0.07 nM. In yet some further embodiments, ketamine in the combinations of the invention may be presented in an amount of about 0.1 nM or less to about 10 nM or more, more specifically, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2 nM, 2.5 nM, 3 nM, 3.5 nM, 4 nM 4.5 nM, 5 nM, 5.5 Nm, 6 nM, 6.5 nM, 7 nM, 7.5 nM, 8 nM, 8.5 nM, 9 nM, 9.5 nM, 10 nM or more. In more specific embodiments, 0.4 nM to about 1.4 nM. In some further embodiments, tianeptine in the combinations of the invention may be presented in an amount of about 0.1 nM or less, to about 80 nM or more, more specifically, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2 nM, 2.5 nM, 3 nM, 3.1 nM, 3.2 nM, 3.3 nM, 3.4 nM, 3.5 nM, 3.6 nM, 3.7 nM, 3.8 nM, 3.9 nM, 4 nM 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, 20 nM, 25 nM, 30 nM, 35 nM, 40 nM, 45 nM, 50 nM, 55 nM, 60 nM, 65 nM, 70 nM, 75 nM, 80 nM or more. In more specific embodiments, 3.2 nM to about 16 nM. These synergistically effective amounts have been converted and adapted herein for use in a mammalian subject, specifically, a human subject. Thus, in some specific and non-limiting embodiments, Buprenorphine may be present in an amount ranging between about 0.0001 mg to about 10 mg or more, specifically, between about 0.001 mg to about 1 mg, more specifically, between about 0.001 mg to about 0.2 mg in said combination. In yet some further embodiments, Ketamine may be present in an amount ranging between about 0.01 mg to about 50 mg, more specifically, ranging between about 0.5 mg to about 50 mg, specifically, 0.35 mg to about 17 mg in the combined composition of the invention. Still further, Tianeptine may be present in an amount ranging between about 0.01 mg to about 100 mg, more specifically, between about 0.25 mg to about 25 mg in said combination.

In some specific and non-limiting embodiments, Buprenorphine, and/or any derivatives thereof or any pharmaceutically acceptable salts, esters, metabolites or prodrugs thereof, may be present in the synergistic combinations and/or combined composition of the invention, in an amount ranging between about 0.0001 mg to about 10 mg. Still further, in some specific embodiments Buprenorphine may be present in an amount ranging between about 0.0001 mg to about 1 mg, or between about 0.0002 mg to about 1 mg, or between about 0.0003 mg to about 1 mg, or between about 0.0004 mg to about 1 mg, or between about 0.0005 mg to about 1 mg, or between about 0.0006 mg to about 1 mg, or between about 0.0007 mg to about 1 mg, or between about 0.0008 mg to about 1 mg, or between about 0.0009 mg to about 1 mg, or between about 0.001 mg to about 1 mg in said combination.

Still further, in some other embodiments, Buprenorphine, and/or any derivatives thereof or any pharmaceutically acceptable salts, esters, metabolites or prodrugs thereof, may be present in the synergistic combinations and/or combined composition of the invention, in an amount ranging between about 0.0001 mg to 10 mg, or about 0.0001 mg to about 9 mg, or about 0.0001 mg to about 8 mg, or about 0.0001 mg to about 7 mg, or about 0.0001 mg to about 6 mg, or about 0.0001 mg to about 5 mg, or about 0.0001 mg to about 4 mg, or about 0.0001 mg to about 3 mg in said combination, about 0.0001 mg to about 2 mg, or about 0.0001 mg to about 1 mg in said combination.

In some other specific embodiments, Buprenorphine, and/or any derivatives thereof or any pharmaceutically acceptable salts, esters, metabolites or prodrugs thereof, may be present in the synergistic combinations and/or combined composition of the invention, in an amount ranging between about 0.001 mg to about 2 mg, 0.001 mg to about 1 mg, 0.001 mg to about 0.9 mg, or about 0.001 mg to about 0.8 mg, or about 0.001 mg to about 0.7 mg, or about 0.001 mg to about 0.6 mg, or about 0.001 mg to about 0.5 mg, or about 0.001 mg to about 0.4 mg, or about 0.001 mg to about 0.3 mg, or about 0.001 mg to about 0.2 mg in said combination. In yet some further embodiments, Ketamine, and/or any derivatives thereof or any pharmaceutically acceptable salts, esters, metabolites or prodrugs thereof, may be present in an amount ranging between about 0.01 mg to about 50 mg, or between about 0.02 mg to about 50 mg, or between about 0.03 mg to about 50 mg, or between about 0.04 mg to about 50 mg, or between about 0.05 mg to about 50 mg, or between about 0.06 mg to about 50 mg, or between about 0.07 mg to about 50 mg, or between about 0.08 mg to about 50 mg, or between about 0.09 mg to about 50 mg, or between about 0.10 mg to about 50 mg, or between about 0.11 mg to about 50 mg, or between about 0.12 mg to about 50 mg, or between about 0.13 mg to about 50 mg, or between about 0.14 mg to about 50 mg, or between about 0.15 mg to about 50 mg, or between about 0.16 mg to about 50 mg, or between about 0.17 mg to about 50 mg, or between about 0.18 mg to about 50 mg, or between about 0.19 mg to about 50 mg, or between about 0.20 mg to about 50 mg, or between about 0.21 mg to about 50 mg, or between about 0.22 mg to about 50 mg, or between about 0.23 mg to about 50 mg, or between about 0.24 mg to about 50 mg, or between about 0.25 mg to about 50 mg, or between about 0.26 mg to about 50 mg, or between about 0.27 mg to about 50 mg, or between about 0.28 mg to about 50 mg, or between about 0.29 mg to about 50 mg, or between about 0.30 mg to about 50 mg, or between about 0.31 mg to about 50 mg, or between about 0.32 mg to about 50 mg, or between about 0.33 mg to about 50 mg, or between about 0.34 mg to about 50 mg, or between about 0.35 mg to about 50 mg, in said combination.

In some other specific embodiments, Ketamine, and/or any derivatives thereof or any pharmaceutically acceptable salts, esters, metabolites or prodrugs thereof, may be present in the synergistic combinations and/or combined composition of the invention, in an amount ranging between about 0.35 mg to about 49 mg, or between 0.35 mg to about 48 mg, or between 0.35 mg to about 47 mg, or between 0.35 mg to about 46 mg, or between 0.35 mg to about 45 mg, or between 0.35 mg to about 44 mg, or between 0.35 mg to about 43 mg, or between 0.35 mg to about 42 mg, or between 0.35 mg to about 41 mg, or between 0.35 mg to about 40 mg, or between 0.35 mg to about 40 mg, or between 0.35 mg to about 39 mg, or between 0.35 mg to about 38 mg, or between 0.35 mg to about 37 mg, or between 0.35 mg to about 36 mg, or between 0.35 mg to about 35 mg, or between 0.35 mg to about 34 mg, or between 0.35 mg to about 33 mg, or between 0.35 mg to about 32 mg, or between 0.35 mg to about 31 mg, or between 0.35 mg to about 30 mg, or between 0.35 mg to about 29 mg, or between 0.35 mg to about 28 mg, or between 0.35 mg to about 27 mg, or between 0.35 mg to about 26 mg, or between 0.35 mg to about 25 mg, or between 0.35 mg to about 24 mg, or between 0.35 mg to about 23 mg, or between 0.35 mg to about 22 mg, or between 0.35 mg to about 21 mg, or between 0.35 mg to about 20 mg, or between 0.35 mg to about 19 mg, or between 0.35 mg to about 18 mg, or more specifically, ranging between about 0.5 mg to about 50 mg, or about 0.35 mg to about 17 mg in the combined composition of the invention.

Still further, Tianeptine, and/or any derivatives thereof or any pharmaceutically acceptable salts, esters, metabolites or prodrugs thereof, may be present in the synergistic combinations and/or combined composition of the invention, in an amount ranging between about 0.01 mg to about 100 mg, or between about 0.02 mg to about 100 mg, or between about 0.03 mg to about 100 mg, or between about 0.04 mg to about 100 mg, or between about 0.05 mg to about 100 mg, or between about 0.06 mg to about 100 mg, or between about 0.07 mg to about 100 mg, or between about 0.08 mg to about 100 mg, or between about 0.09 mg to about 100 mg, or between about 0.10 mg to about 100 mg, or between about 0.11 mg to about 100 mg, or between about 0.12 mg to about 100 mg, or between about 0.13 mg to about 100 mg, or between about 0.14 mg to about 100 mg, or between about 0.15 mg to about 100 mg, or between about 0.16 mg to about 100 mg, or between about 0.17 mg to about 100 mg, or between about 0.18 mg to about 100 mg, or between about 0.19 mg to about 100 mg, or between about 0.20 mg to about 100 mg, or between about 0.2 lmg to about 100 mg, or between about 0.22 mg to about 100 mg, or between about 0.23 mg to about 100 mg, or between about 0.24 mg to about 100 mg, or between about 0.25 mg to about 100 mg in said combination. In some other specific embodiments, Tianeptine, and/or any derivatives thereof or any pharmaceutically acceptable salts, esters, metabolites or prodrugs thereof, may be present in the synergistic combinations and/or combined composition of the invention, in an amount ranging between about 0.25 mg to about 95 mg, or between about 0.25 mg to about 90 mg, or between about 0.25 mg to about 85 mg, or between about 0.25 mg to about 80 mg, or between about 0.25 mg to about 75 mg, or between about 0.25 mg to about 70 mg, or between about 0.25 mg to about 65 mg, or between about 0.25 mg to about 60 mg, or between about 0.25 mg to about 55 mg, or between about 0.25 mg to about 50 mg, or between about 0.25 mg to about 49 mg, or between about 0.25 mg to about 48 mg, or between about 0.25 mg to about 47 mg, or between about 0.25 mg to about 46 mg, or between about 0.25 mg to about 45 mg, or between about 0.25 mg to about 44 mg, or between about 0.25 mg to about 43 mg, or between about 0.25 mg to about 42 mg, or between about 0.25 mg to about 41 mg, or between about 0.25 mg to about 40 mg, or between about 0.25 mg to about 39 mg, or between about 0.25 mg to about 38 mg, or between about 0.25 mg to about 37 mg, or between about 0.25 mg to about 36 mg, or between about 0.25 mg to about 35 mg, or between about 0.25 mg to about 34 mg, or between about 0.25 mg to about 33 mg, or between about 0.25 mg to about 32 mg, or between about 0.25 mg to about 31 mg, or between about 0.25 mg to about 30 mg, or between about 0.25 mg to about 29 mg, or between about 0.25 mg to about 28 mg, or between about 0.25 mg to about 27 mg, or between about 0.25 mg to about 26 mg, or more specifically, between about 0.25 mg to about 25 mg in said combination.

It should be understood that the MOR modulators amounts indicated herein refer in some embodiments, to the amount per one dose, or per each dose, in case more than one dose is administered.

In yet some further embodiments, the combined composition of the invention is a composition adapted for daily administration. Thus, in some embodiments, the combined composition of the invention may be referred to as a daily composition.

It should be appreciated that the amount used in the combined composition for each modulator varies and depend on the administration mode used, and the specific disorder treated. Thus, in some embodiments that will be further described in more detail herein after, the combined compositions of the invention may be used treating, preventing, ameliorating, reducing or delaying the onset of at least one of suicidality, a mental disorder and a physical pain in a subject in need thereof. As such, the combined compositions of the invention may be in some embodiments, formulated as pharmaceutical compositions.

As noted above, the pharmaceutical combined composition of the invention may optionally further comprise at least one pharmaceutically acceptable carrier/s, excipient/s, auxiliaries, and/or diluent/s. “Pharmaceutically or therapeutically acceptable carrier” refers to a carrier medium which does not interfere with the effectiveness of the biological activity of the active ingredients. As mentioned herein, the compositions provided by the invention optionally further comprise at least one pharmaceutically acceptable excipient or carrier. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

As used herein “pharmaceutically acceptable carrier/diluents/excipient” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

In various embodiments, the final solution of any of the compositions of the invention may be adjusted with a pharmacologically acceptable acid, base or buffer.

In some embodiments, the synergistic combinations and/or compositions of the invention may be suitable for systemic administration. The pharmaceutical composition of the invention can be administered and dosed by the methods of the invention, in accordance with good medical practice. More specifically, the compositions used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers (MDI's), or dry powder dispersion devices.

In some specific embodiments, the administration may be intranasal e.g. by administration of drops applied to the nasal mucosa. In some other embodiments, the administration may be sublingual e.g. by administration of tablets or lozenges that the subject keeps under the tongue until they dissolve completely (without chewing, sucking or swallowing).

In other embodiments the pharmaceutical composition may be adapted for topical administration. By “topical administration” it is meant that the pharmaceutical composition and the carrier may be adapted to any mode of topical administration including: epicutaneous, transdermal, oral, bronchoalveolar lavage, ophtalmic administration, enema, nasal administration, administration to the ear, administration by inhalation.

Regardless of the route of administration selected, the compositions of the present invention, which may be used in a suitable hydrated form, and/or the pharmaceutical compositions of the present invention, are formulated into pharmaceutically-acceptable dosage forms by conventional methods known to those of skill in the art.

Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, and suitable mixtures thereof. The proper fluidity can be maintained, for example, by the use of a coating, such as lecithin, by the maintenance of the required particle size in the case of dispersion and by the use of surfactants.

It should be understood that although the MOR modulator-combinations of the invention are described herein as comprised within combined compositions and kits, the invention also pertains to any one of the synergistic combinations described by the invention. Thus, a further aspect of the invention relates to synergistic combinations of MOR modulators, specifically, modulators that synergistically activate MOR. In more specific embodiments, the synergistic combinations of the invention may comprise at least two of buprenorphine, ketamine and tianeptine. It should be noted that any MOR modulator disclosed by the invention herein before in connection with other aspects of the invention, may be also applicable for the synergistic combinations of the invention as described herein. In yet some further embodiments, the invention further provides combinations comprising at least two of Dezocine, morphine, propoxyphene, Oxycodone, Fentanyl, Heroin, Hydrocodone, Hydromorphone, Levorphanol, Meperidine, Methadone, Oxymorphone, Butorphanol, Pentazocine, Tramadol, Mitragynine and Codeine any derivatives thereof, or any pharmaceutically acceptable salts, esters, metabolites or prodrugs thereof. The invention further provides the use of these combinations for any of the therapeutic applications disclosed herein after.

As noted above, the present invention involves the use of different active ingredients, specifically, at least two MOR modulators, for example, the buprenorphine, ketamine and tianeptine, that may be administered through different routes, dosages and combinations. More specifically, the treatment of suicidality, mental disorders, mental pain and physical pain as well as any conditions associated therewith, with a combination of active ingredients may involve separate administration of each active ingredient. Therefore, a kit providing a convenient modular format for the combined therapy using the MOR modulators of the invention, specifically, the synergistic MOR activators, buprenorphine, ketamine and tianeptine, required for treatment, would allow the desired or preferred flexibility in the above parameters.

Thus, in another aspect, the invention provides a kit comprising at least two MOR modulators. In some optional embodiments, each MOR modulator of the kit of the invention, may be provided in a pharmaceutical dosage form. It should be noted that the kit may optionally further comprise container means for containing said dosage forms.

In some embodiments, the at least two MOR modulators of the kit of the invention synergistically activate MOR.

In some specific embodiments, the MOR modulators of the kit of the invention may comprise at least one of Buprenorphine, Ketamine, Tianeptine, morphine, propoxyphene, oxycodone, Fentanyl, Heroin, Hydrocodone, Hydromorphone, Levorphanol, Meperidine, Methadone, Oxymorphone, Butorphanol, Pentazocine, Tramadol, Codeine, Mitragynine and Dezocine, any derivatives thereof, or any pharmaceutically acceptable salts, esters, metabolites or prodrugs thereof.

It should be appreciated that any of the MOR modulators disclosed by the invention herein before in connection with other aspects of the invention, may be also applicable for the kits of the invention as described herein.

In some particular embodiments, the kit of the invention may comprise:

(a) an effective amount of buprenorphine, any derivatives thereof, or any pharmaceutically acceptable salts, esters, metabolites or prodrugs thereof, optionally in a first pharmaceutical dosage form; (b) an effective amount of ketamine, any derivatives thereof, or any pharmaceutically acceptable salts, esters, metabolites or prodrugs thereof, optionally in a second pharmaceutical dosage form. In some embodiments, the kit of the invention may optionally further comprise (c), container means for containing said first and second dosage forms.

In yet some further specific embodiments, the kit of the invention may comprise:

(a) an effective amount of buprenorphine any derivatives thereof or any pharmaceutically acceptable salts, esters, metabolites or prodrugs thereof, optionally in a first pharmaceutical dosage form; and (b) an effective amount of tianeptine any derivatives thereof or any pharmaceutically acceptable salts, esters, metabolites or prodrugs thereof, optionally in a second pharmaceutical dosage form. In some embodiments, the kit of the invention may optionally further comprise (c), container means for containing said first and second dosage forms.

Still further, in some embodiments the kit of the invention may comprise:

(a) an effective amount of ketamine any derivatives thereof or any pharmaceutically acceptable salts, esters, metabolites or prodrugs thereof, optionally in a first pharmaceutical dosage form; and
(b) an effective amount of tianeptine, any derivatives thereof or any pharmaceutically acceptable salts, esters, metabolites or prodrugs thereof, optionally in a second pharmaceutical dosage form. In some embodiments, the kit of the invention may optionally further comprise (c), container means for containing said first and second dosage forms.

In some further embodiments, the kit of the invention may comprise:

(a) an effective amount of buprenorphine, any derivatives thereof or any pharmaceutically acceptable salts, esters, metabolites or prodrugs thereof, optionally in a first pharmaceutical dosage form; (b) an effective amount of tianeptine, any derivatives thereof or any pharmaceutically acceptable salts, esters, metabolites or prodrugs thereof, optionally in a second pharmaceutical dosage form; (c) an effective amount of ketamine any derivatives thereof or any pharmaceutically acceptable salts, esters, metabolites or prodrugs thereof, optionally in a third pharmaceutical dosage form. In some embodiments, the kit of the invention may optionally further comprise (d), container means for containing said first, second and third dosage forms.

In yet some specific embodiments, the kits of the invention may comprise Buprenorphine, and/or any derivatives thereof or any pharmaceutically acceptable salts, esters, metabolites or prodrugs thereof, in an amount ranging between about 0.0001 mg to about 1 mg, specifically, between about 0.001 mg to about 0.2 mg. In yet some further embodiments, Ketamine may be present in an amount ranging between about 0.01 mg to about 50 mg, more specifically, ranging between about 0.5 mg to about 50 mg, ranging between about 0.35 mg to about 17 mg in the kits of the invention. Still further, Tianeptine may be present in an amount ranging between about 0.01 mg to about 100 mg, more specifically, between about 0.25 mg to about 25 mg in the kits of the invention.

In yet some specific embodiments, the kits of the invention may comprise Buprenorphine, and/or any derivatives thereof or any pharmaceutically acceptable salts, esters, metabolites or prodrugs thereof, in an amount ranging between about 0.0001 mg to about 10 mg. In yet some further embodiments, the kits of the invention may comprise Buprenorphine, and/or any derivatives thereof or any pharmaceutically acceptable salts, esters, metabolites or prodrugs thereof, in an amount ranging between about 0.0001 mg to about 1 mg, or between about 0.0002 mg to about 1 mg, or between about 0.0003 mg to about 1 mg, or between about 0.0004 mg to about 1 mg, or between about 0.0005 mg to about 1 mg, or between about 0.0006 mg to about 1 mg, or between about 0.0007 mg to about 1 mg, or between about 0.0008 mg to about 1 mg, or between about 0.0009 mg to about 1 mg, or between about 0.001 mg to about 1 mg.

In some other embodiments, the kits of the invention may comprise Buprenorphine, and/or any derivatives thereof or any pharmaceutically acceptable salts, esters, metabolites or prodrugs thereof, in an amount ranging between about 0.0001 mg to 10 mg, or about 0.0001 mg to about 9 mg, or about 0.0001 mg to about 8 mg, or about 0.0001 mg to about 7 mg, or about 0.0001 mg to about 6 mg, or about 0.0001 mg to about 5 mg, or about 0.0001 mg to about 4 mg, or about 0.0001 mg to about 3 mg in said combination, or about 0.0001 mg to about 2 mg in said combination.

In some other embodiments, the kits of the invention may comprise Buprenorphine, and/or any derivatives thereof or any pharmaceutically acceptable salts, esters, metabolites or prodrugs thereof, in an amount ranging between about 0.001 mg to about 0.9 mg, or about 0.001 mg to about 0.8 mg, or about 0.001 mg to about 0.7 mg, or about 0.001 mg to about 0.6 mg, or about 0.001 mg to about 0.5 mg, or about 0.001 mg to about 0.4 mg, or about 0.001 mg to about 0.3 mg, or about 0.001 mg to about 0.2 mg.

In yet some specific embodiments, the kits of the invention may comprise Ketamine, and/or any derivatives thereof or any pharmaceutically acceptable salts, esters, metabolites or prodrugs thereof, in an amount ranging between about 0.01 mg to about 50 mg, or between about 0.02 mg to about 50 mg, or between about 0.03 mg to about 50 mg, or between about 0.04 mg to about 50 mg, or between about 0.05 mg to about 50 mg, or between about 0.06 mg to about 50 mg, or between about 0.07 mg to about 50 mg, or between about 0.08 mg to about 50 mg, or between about 0.09 mg to about 50 mg, or between about 0.10 mg to about 50 mg, or between about 0.11 mg to about 50 mg, or between about 0.12 mg to about 50 mg, or between about 0.13 mg to about 50 mg, or between about 0.14 mg to about 50 mg, or between about 0.15 mg to about 50 mg, or between about 0.16 mg to about 50 mg, or between about 0.17 mg to about 50 mg, or between about 0.18 mg to about 50 mg, or between about 0.19 mg to about 50 mg, or between about 0.20 mg to about 50 mg, or between about 0.21 mg to about 50 mg, or between about 0.22 mg to about 50 mg, or between about 0.23 mg to about 50 mg, or between about 0.24 mg to about 50 mg, or between about 0.25 mg to about 50 mg, or between about 0.26 mg to about 50 mg, or between about 0.27 mg to about 50 mg, or between about 0.28 mg to about 50 mg, or between about 0.29 mg to about 50 mg, or between about 0.30 mg to about 50 mg, or between about 0.31 mg to about 50 mg, or between about 0.32 mg to about 50 mg, or between about 0.33 mg to about 50 mg, or between about 0.34 mg to about 50 mg, or between about 0.35 mg to about 50 mg in said combination.

In some other embodiments, the kits of the invention may comprise Ketamine, and/or any derivatives thereof or any pharmaceutically acceptable salts, esters, metabolites or prodrugs thereof, in an amount ranging between 0.35 mg to about 49 mg, or between 0.35 mg to about 48 mg, or between 0.35 mg to about 47 mg, or between 0.35 mg to about 46 mg, or between 0.35 mg to about 45 mg, or between 0.35 mg to about 44 mg, or between 0.35 mg to about 43 mg, or between 0.35 mg to about 42 mg, or between 0.35 mg to about 41 mg, or between 0.35 mg to about 40 mg, or between 0.35 mg to about 40 mg, or between 0.35 mg to about 39 mg, or between 0.35 mg to about 38 mg, or between 0.35 mg to about 37 mg, or between 0.35 mg to about 36 mg, or between 0.35 mg to about 35 mg, or between 0.35 mg to about 34 mg, or between 0.35 mg to about 33 mg, or between 0.35 mg to about 32 mg, or between 0.35 mg to about 31 mg, or between 0.35 mg to about 30 mg, or between 0.35 mg to about 29 mg, or between 0.35 mg to about 28 mg, or between 0.35 mg to about 27 mg, or between 0.35 mg to about 26 mg, or between 0.35 mg to about 25 mg, or between 0.35 mg to about 24 mg, or between 0.35 mg to about 23 mg, or between 0.35 mg to about 22 mg, or between 0.35 mg to about 21 mg, or between 0.35 mg to about 20 mg, or between 0.35 mg to about 19 mg, or between 0.35 mg to about 18 mg, specifically, between about 0.5 mg to about 50 mg, or more specifically, ranging between about 0.35 mg to about 17 mg.

In yet some specific embodiments, the kits of the invention may comprise Tianeptine, and/or any derivatives thereof or any pharmaceutically acceptable salts, esters, metabolites or prodrugs thereof, in an amount ranging between 0.01 mg to about 100 mg, or between about 0.02 mg to about 100 mg, or between about 0.03 mg to about 100 mg, or between about 0.04 mg to about 100 mg, or between about 0.05 mg to about 100 mg, or between about 0.06 mg to about 100 mg, or between about 0.07 mg to about 100 mg, or between about 0.08 mg to about 100 mg, or between about 0.09 mg to about 100 mg, or between about 0.10 mg to about 100 mg, or between about 0.11 mg to about 100 mg, or between about 0.12 mg to about 100 mg, or between about 0.13 mg to about 100 mg, or between about 0.14 mg to about 100 mg, or between about 0.15 mg to about 100 mg, or between about 0.16 mg to about 100 mg, or between about 0.17 mg to about 100 mg, or between about 0.18 mg to about 100 mg, or between about 0.19 mg to about 100 mg, or between about 0.20 mg to about 100 mg, or between about 0.21 mg to about 100 mg, or between about 0.22 mg to about 100 mg, or between about 0.23 mg to about 100 mg, or between about 0.24 mg to about 100 mg, or between about 0.25 mg to about 100 mg.

In some other embodiments, the kits of the invention may comprise Tianeptine, and/or any derivatives thereof or any pharmaceutically acceptable salts, esters, metabolites or prodrugs thereof, in an amount ranging between or between about 0.25 mg to about 95 mg, or between about 0.25 mg to about 90 mg, or between about 0.25 mg to about 85 mg, or between about 0.25 mg to about 80 mg, or between about 0.25 mg to about 75 mg, or between about 0.25 mg to about 70 mg, or between about 0.25 mg to about 65 mg, or between about 0.25 mg to about 60 mg, or between about 0.25 mg to about 55 mg, or between about 0.25 mg to about 50 mg, or between about 0.25 mg to about 49 mg, or between about 0.25 mg to about 48 mg, or between about 0.25 mg to about 47 mg, or between about 0.25 mg to about 46 mg, or between about 0.25 mg to about 45 mg, or between about 0.25 mg to about 44 mg, or between about 0.25 mg to about 43 mg, or between about 0.25 mg to about 42 mg, or between about 0.25 mg to about 41 mg, or between about 0.25 mg to about 40 mg, or between about 0.25 mg to about 39 mg, or between about 0.25 mg to about 38 mg, or between about 0.25 mg to about 37 mg, or between about 0.25 mg to about 36 mg, or between about 0.25 mg to about 35 mg, or between about 0.25 mg to about 34 mg, or between about 0.25 mg to about 33 mg, or between about 0.25 mg to about 32 mg, or between about 0.25 mg to about 31 mg, or between about 0.25 mg to about 30 mg, or between about 0.25 mg to about 29 mg, or between about 0.25 mg to about 28 mg, or between about 0.25 mg to about 27 mg, or between about 0.25 mg to about 26 mg, or more specifically, between about 0.25 mg to about 25 mg.

It should be understood that the amounts indicated herein for the kits of the invention are applicable for one dose of the synergistic combinations of the invention. In some embodiments, such dose may be useful for a daily administration, for example, one administration of one dose per day or two administrations of one dose, or alternatively two doses per day. Thus, in some embodiments, the kits of the invention is referred to herein as a daily kit.

Thus, it should be appreciated that the kits of the invention may comprise at least one dose of the synergistic combinations of the invention, and in some embodiments, at least two, at least three, at least four, at least six, at least seven, at least eight, at least nine, at least ten or more doses of the synergistic combinations of the invention. In yet some further embodiments, the kits of the invention may comprise between 1 to 100 doses of the combination of the invention, wherein each dose contain the amounts of the active ingredients specified above. More specifically, 1, 2, 3, 4, 5, 6, 7, 8, 9, 10, 11, 12, 13, 14, 1, 16, 17, 18, 19, 20, 25, 30, 35, 40, 45, 50, 55, 60, 65, 70, 75, 80, 85, 90, 95, 100 or more doses of the synergistic combinations of MOR modulators disclosed by the invention.

In yet some further embodiments, the invention provides kits for use in a method of treating, preventing, ameliorating, reducing or delaying the onset of at least one of suicidality, a mental disorder and a physical pain in a subject in need thereof. Detailed description of each of the relevant pathologic conditions, is disclosed herein after in connection with the therapeutic methods provided by the invention. It should be appreciated that this description is also relevant in connection with any aspect of the invention, specifically, the kits, the synergistic combinations, the combined compositions and uses disclosed by the invention.

In more specific embodiments, the kit of the invention may be used in a method of treating, preventing, ameliorating, reducing acute suicidality in a subject in need.

In yet some further embodiments, the kits of the invention may be used in a method of treating, preventing, ameliorating, reducing or delaying the onset of at least one mental disorder. In more specific embodiments, such mental disorder may be at least one of mood disorder, psychotic disorder, personality disorder and anxiety disorder.

In more specific embodiments, the kits of the invention may be suitable for treating at least one mood disorder, more specifically, at least one of mental pain, major depression, treatment resistant depression (TRD), mania, and bipolar disorders. In yet some further embodiments, the kits of the invention may be applicable for treating at least one psychotic disorder, for example, at least one of schizophrenia, schizoaffective disorder, schizophreniform disorder, delusional disorder, brief psychotic disorder, shared psychotic disorder, substance-induced psychotic disorder, psychotic disorder due to a medical condition and paraphrenia. Still further in some embodiments, the kits of the invention may be applicable for personality disorders, specifically, at least one of paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive disorder. In certain embodiments, the kits of the invention may be used for treating at least one anxiety disorder, specifically, at least one of panic disorder, generalized anxiety disorder (GAD), specific phobia, social anxiety disorder (SAD), separation anxiety disorder, agoraphobia, panic disorder, and selective mutism.

In more specific embodiments, the invention provides kits for use in methods of treating, preventing, ameliorating, reducing or delaying the onset of at least one of mental pain and depression. It should be noted that “mental disorder” or “mental illness” or “mental disease” or “psychiatric or neuropsychiatric disease or illness or disorder” refers to mood disorders, psychotic disorders, personality disorders and anxiety disorders as well as other mental disorders such as substance-related disorders, childhood disorders, dementia, autistic disorder, adjustment disorder, delirium, multi-infarct dementia, and Tourette's disorder.

In yet some further embodiments, the kit of the invention may be used for treating physical pain, as defined herein after, in connection with the therapeutic methods of the invention.

According to some embodiments, the kit of the invention may further comprise container means for containing the different components of the kit of the invention or any dosage forms thereof. The term “container” as used herein refers to any receptacle capable of holding at least one component of a pharmaceutical composition of the invention. Such a container may be any jar, vial or box known to a person skilled in the art and may be made of any material suitable for the components contained therein and additionally suitable for short or long term storage under any kind of temperature. More specifically, the kit includes container means for containing separate compositions; such as a divided bottle or a divided foil packet however, the separate compositions may also be contained within a single, undivided container. Typically, the kit includes directions for the administration of the separate components, compounds or agents. As noted above, the kit form is particularly advantageous when the separate components, compounds or agents are preferably administered in different dosage forms (e.g., oral and parenteral), are administered at different dosage intervals, or when titration of the individual components of the combination is desired by the prescribing physician.

In some more specific embodiments, the invention provides a kit comprising (a) an effective amount of buprenorphine, any derivatives thereof, or any pharmaceutically acceptable salts, esters, metabolites or prodrugs thereof, optionally in a first pharmaceutical dosage form; (b) an effective amount of ketamine, any derivatives thereof, or any pharmaceutically acceptable salts, esters, metabolites or prodrugs thereof, optionally in a second pharmaceutical dosage form. In some embodiments, the kit of the invention may optionally further comprise (c), container means for containing said first and second dosage forms. In more specific embodiments, the kit may be for use in a method of treating, preventing, ameliorating, reducing suicidality in a subject in need. In yet other specific embodiments, the invention provides a kit comprising (a) an effective amount of ketamine, any derivatives thereof, or any pharmaceutically acceptable salts, esters, metabolites or prodrugs thereof, optionally in a first pharmaceutical dosage form; (b) an effective amount of buprenorphine, any derivatives thereof, or any pharmaceutically acceptable salts, esters, metabolites or prodrugs thereof, optionally in a second pharmaceutical dosage form. In some embodiments, the kit of the invention may optionally further comprise (c), container means for containing said first and second dosage forms. In more specific embodiments, the kit may be for use in a method of treating, preventing, ameliorating, reducing depression in a subject in need. In yet other specific embodiments, the invention provides a kit comprising (a) an effective amount of ketamine, any derivatives thereof, or any pharmaceutically acceptable salts, esters, metabolites or prodrugs thereof, optionally in a first pharmaceutical dosage form; (b) an effective amount of tianeptine, any derivatives thereof, or any pharmaceutically acceptable salts, esters, metabolites or prodrugs thereof, optionally in a second pharmaceutical dosage form. In some embodiments, the kit of the invention may optionally further comprise (c), container means for containing said first and second dosage forms. In more specific embodiments, the kit may be for use in a method of treating, preventing, ameliorating, reducing physical pain in a subject in need.

In yet some further aspect thereof, the invention provides methods for activating MOR in a cell. More specifically, the method of the invention may comprise the steps of contacting said cell with an effective amount of at least two MOR modulators, specifically, any of the activating modulators discussed by the invention, and combinations thereof, any combined compositions thereof or any kits comprising the at least two activating MOR modulators of the invention. In more specific embodiments, the methods of the invention may comprise the step of contacting the cell with an effective amount of at least two of buprenorphine, ketamine and tianeptine.

Still further, the invention provides methods for activating MOR in a cell of a subject in need thereof. In some specific embodiments, the method of the invention may comprise the step of administering the subject with an activating effective amount of at least two MOR modulators as discussed by the invention. In some specific embodiments, the methods of the invention may comprise the step of administering to the subject, an effective amount of at least two of Buprenorphine, Ketamine, Tianeptine, Dezocine, morphine, propoxyphene, Oxycodone, Fentanyl, Heroin, Hydrocodone, Hydromorphone, Levorphanol, Meperidine, Methadone, Oxymorphone, Butorphanol, Pentazocine, Tramadol, Mitragynine and Codeine any derivatives thereof, or any pharmaceutically acceptable salts, esters, metabolites or prodrugs thereof, more specifically, at least two of buprenorphine, ketamine and tianeptine, any synergistic combination or mixture thereof, any combined composition or any kit comprising the combined MOR modulators of the invention.

It should be appreciated that the invention further encompasses the use of at least two of the modulators of the invention, specifically, buprenorphine, ketamine and tianeptine, in methods for synergistically activating MOR, in a cell of a subject in need.

The invention describes combinations of MOR modulators that synergistically activate MOR. Since activation of MOR has been previously shown as having a positive effect of suicidality and depression, the novel synergistic combinations of the invention may display enhanced effects on these conditions and may offer an effective approach for significantly reducing the amount of MOR modulators required for addressing these pathologic conditions. The combined compositions, combinations and kits of the invention may be successfully used for therapeutic applications.

Thus, another aspect of the invention relates to a method of treating, preventing, ameliorating, reducing or delaying the onset of at least one of suicidality, a mental disorder and a physical pain in a subject in need thereof. In more specific embodiments, the methods of the invention may comprise the step of administering to the subject a therapeutically effective amount of at least two MOR modulators, any combinations thereof, any combined compositions thereof or any kit comprising the same, specifically, the combinations of the invention.

In some embodiments, the at least two MOR modulators used by the methods of the invention synergistically activate MOR.

In some specific embodiments, MOR modulators, specifically, positive modulators that activate MOR, that may be used in the methods of the invention may be at least one of buprenorphine, ketamine, tianeptine, dezocine, morphine, propoxyphene, Fentanyl, Heroin, Hydrocodone, Hydromorphone, Levorphanol, Meperidine, Methadone, Oxymorphone, Butorphanol, Pentazocine, Tramadol, Codeine, Mitragynine and Oxycodone, any derivatives thereof or any pharmaceutically acceptable salts, esters, metabolite or prodrugs thereof.

It should be appreciated that any of the MOR modulators disclosed by the invention herein before in connection with other aspects of the invention, may be also applicable for the methods of the invention as described herein.

In some specific embodiments, the methods of the invention may involve the step of administering two MOR modulators, specifically, two activating modulators of MOR.

In some specific embodiments, the invention provides a therapeutic method comprising the step of administering to the subject a therapeutically effective amount of buprenorphine and ketamine, any derivatives thereof or any pharmaceutically acceptable salts, esters or prodrugs thereof.

In yet some further specific embodiments, the invention provides a therapeutic method comprising the step of administering to the subject a therapeutically effective amount of buprenorphine and tianeptine, any derivatives thereof or any pharmaceutically acceptable salts, esters, metabolite or prodrugs thereof.

In some alternative embodiments, the invention provides a therapeutic method comprising the step of administering to the subject a therapeutically effective amount of ketamine and tianeptine, any derivatives thereof or any pharmaceutically acceptable salts, esters, metabolite or prodrugs thereof. In yet some further specific embodiments, the methods of the invention may involve the step of administering three MOR modulators, specifically, three activating modulators of MOR.

Thus, in some specific embodiments, the invention provides a therapeutic method comprising the step of administering to the subject a therapeutically effective amount of buprenorphine, ketamine and tianeptine, any derivatives thereof or any pharmaceutically acceptable salts, esters, metabolite or prodrugs thereof.

For enhancing the synergistic effect of the combined therapy provided by the invention, in some embodiments, buprenorphine, and/or any derivatives thereof or any pharmaceutically acceptable salts, esters, metabolites or prodrugs thereof, may be administered to the subject in an amount ranging between about 0.000001 mg/kg to about 0.1 mg/kg, per day, or 0.000001 mg/kg to about 0.01 mg/kg, per day, specifically, between about 0.00001 mg/kg to about 0.001 mg/kg, per day. In yet some further embodiments ketamine, and/or any derivatives thereof or any pharmaceutically acceptable salts, esters, metabolites or prodrugs thereof, may be administered by the methods of the invention in an amount ranging between about 0.001 mg/kg to about 1 mg/kg, per day, specifically, in an amount ranging between about 0.005 mg/kg to about 0.5 mg/kg, per day. In some further embodiments, tianeptine, and/or any derivatives thereof or any pharmaceutically acceptable salts, esters, metabolites or prodrugs thereof, may be administered by the methods of the invention in an amount ranging between about 0.001 mg/kg to about 1.5 mg/kg, specifically, in an amount ranging between about 0.0035 mg/kg to about 0.35 mg/kg.

For enhancing the synergistic effect of the combined therapy provided by the invention, in some embodiments, Buprenorphine, and/or any derivatives thereof or any pharmaceutically acceptable salts, esters, metabolites or prodrugs thereof, may be administered to the subject in an amount ranging between about 0.000001 mg/kg to about 0.01 mg/kg, per day, or between about 0.000002 mg/kg to about 0.01 mg/kg, or between about 0.000003 mg/kg to about 0.01 mg/kg, or between about 0.000004 mg/kg to about 0.01 mg/kg, or between about 0.000005 mg/kg to about 0.01 mg/kg, or between about 0.000006 mg/kg to about 0.01 mg/kg, or between about 0.000007 mg/kg to about 0.01 mg/kg, or between about 0.000008 mg/kg to about 0.01 mg/kg, or between about 0.000009 mg/kg to about 0.01 mg/kg, or between about 0.00001 mg/kg to about 0.01 mg/kg.

In some other embodiments, Buprenorphine, and/or any derivatives thereof or any pharmaceutically acceptable salts, esters, metabolites or prodrugs thereof, may be administered to the subject in an amount ranging between about 0.00001 mg/kg to about 0.001 mg/kg, per day, or between about 0.00001 mg/kg to about 0.002 mg/kg, or between about 0.00001 mg/kg to about 0.003 mg/kg, or between about 0.00001 mg/kg to about 0.004 mg/kg, or between about 0.00001 mg/kg to about 0.005 mg/kg, or between about 0.00001 mg/kg to about 0.006 mg/kg, or between about 0.00001 mg/kg to about 0.008 mg/kg, or between about 0.00001 mg/kg to about 0.009 mg/kg.

In some further embodiments, Buprenorphine, and/or any derivatives thereof or any pharmaceutically acceptable salts, esters, metabolites or prodrugs thereof, may be administered to the subject in an amount ranging between about 0.00001 mg/kg to about 0.1 mg/kg per day, or between about 0.000002 mg/kg to about 0.1 mg/kg, or between about 0.000003 mg/kg to about 0.1 mg/kg, or between about 0.000004 mg/kg to about 0.1 mg/kg, or between about 0.000005 mg/kg to about 0.1 mg/kg, or between about 0.000006 mg/kg to about 0.1 mg/kg, or between about 0.000007 mg/kg to about 0.1 mg/kg, or between about 0.000008 mg/kg to about 0.1 mg/kg, or between about 0.000009 mg/kg to about 0.1 mg/kg, or between about 0.00001 mg/kg to about 0.1 mg/kg.

In some further embodiments, Ketamine, and/or any derivatives thereof or any pharmaceutically acceptable salts, esters, metabolites or prodrugs thereof, may be administered to the subject in an amount ranging between about 0.001 mg/kg to about 1 mg/kg, per day, or between about 0.002 mg/kg to about 1 mg/kg, or between about 0.003 mg/kg to about 1 mg/kg, or between about 0.005 mg/kg to about 1 mg/kg, or between about 0.005 mg/kg to about 0.5 mg/kg.

In some other embodiments, Ketamine, and/or any derivatives thereof or any pharmaceutically acceptable salts, esters, metabolites or prodrugs thereof, may be administered specifically, in an amount ranging between about 0.005 mg/kg to about 0.5 mg/kg, per day, or between 0.05 mg/kg to about 0.3 mg/kg, or between 0.05 mg/kg to about 0.3 mg/kg, or between 0.05 mg/kg to about 0.35 mg/kg, or between 0.05 mg/kg to about 0.4 mg/kg, or between 0.05 mg/kg to about 0.45 mg/kg, or between 0.5 mg/kg to about 0.3 mg/kg, or between 0.05 mg/kg to about 0.55 mg/kg, or between 0.05 mg/kg to about 0.6 mg/kg, or between 0.05 mg/kg to about 0.65 mg/kg, or between 0.05 mg/kg to about 0.7 mg/kg, or between 0.05 mg/kg to about 0.75 mg/kg, or between 0.05 mg/kg to about 0.8 mg/kg, or between 0.05 mg/kg to about 0.85 mg/kg, or between 0.05 mg/kg to about 0.9 mg/kg, or between 0.05 mg/kg to about 0.95 mg/kg to the subject.

In some further embodiments, Tianeptine, and/or any derivatives thereof or any pharmaceutically acceptable salts, esters, metabolites or prodrugs thereof, may be administered by the methods of the invention in an amount ranging between about 0.001 mg/kg to about 1.5 mg/kg, per day, or between about 0.001 mg/kg to about 1.5 mg/kg, or between about 0.002 mg/kg to about 1.5 mg/kg, or between about 0.003 mg/kg to about 1.5 mg/kg, or between about 0.004 mg/kg to about 1.5 mg/kg, or between about 0.005 mg/kg to about 1.5 mg/kg, or between about 0.006 mg/kg to about 1.5 mg/kg, or between about 0.007 mg/kg to about 1.5 mg/kg, or between about 0.008 mg/kg to about 1.5 mg/kg, or between about 0.009 mg/kg to about 1.5 mg/kg, or between about 0.01 mg/kg to about 1.5 mg/kg, or between about 0.02 mg/kg to about 1.5 mg/kg, or between about 0.03 mg/kg to about 1.5 mg/kg, or between about 0.04 mg/kg to about 1.5 mg/kg, or between about 0.05 mg/kg to about 1.5 mg/kg, or between about 0.06 mg/kg to about 1.5 mg/kg, or between about 0.07 mg/kg to about 1.5 mg/kg, or between about 0.08 mg/kg to about 1.5 mg/kg, or between about 0.09 mg/kg to about 1.5 mg/kg, or between about 0.1 mg/kg to about 1.5 mg/kg, or between about 0.2 mg/kg to about 1.5 mg/kg, or between about 0.3 mg/kg to about 1.5 mg/kg, or between about 0.4 mg/kg to about 1.5 mg/kg, or between about 0.5 mg/kg to about 1.5 mg/kg. Alternatively, between about 0.030 mg/kg to about 1.5 mg/kg, or more specifically, between about 0.035 mg/kg to about 1.5 mg/kg to the subject.

In some further embodiments, Tianeptine, and/or any derivatives thereof or any pharmaceutically acceptable salts, esters, metabolites or prodrugs thereof, may specifically be administered by the methods of the invention in an amount ranging between about 0.0035 mg/kg to about 0.35 mg/kg, per day, or between about 0.0035 mg/kg to about 0.3 mg/kg, or between about 0.0035 mg/kg to about 0.35 mg/kg, or between about 0.0035 mg/kg to about 0.40 mg/kg, or between about 0.0035 mg/kg to about 0.45 mg/kg, or between about 0.0035 mg/kg to about 0.5 mg/kg, or between about 0.0035 mg/kg to about 0.55 mg/kg, or between about 0.0035 mg/kg to about 0.6 mg/kg, or between about 0.0035 mg/kg to about 0.65 mg/kg, or between about 0.0035 mg/kg to about 0.70 mg/kg, or between about 0.0035 mg/kg to about 0.75 mg/kg, or between about 0.0035 mg/kg to about 0.8 mg/kg, or between about 0.0035 mg/kg to about 0.85 mg/kg, or between about 0.0035 mg/kg to about 0.9 mg/kg, or between about 0.0035 mg/kg to about 0.95 mg/kg, or between about 0.0035 mg/kg to about 1 mg/kg, or between about 0.0305 mg/kg to about 1.05 mg/kg, or between about 0.0035 mg/kg to about 1.1 mg/kg, or between about 0.0035 mg/kg to about 1.15 mg/kg, or between about 0.0035 mg/kg to about 1.2 mg/kg, or between about 0.0035 mg/kg to about 1.25 mg/kg, or between about 0.0035 mg/kg to about 1.3 mg/kg, or between about 0.0035 mg/kg to about 1.35 mg/kg, or between about 0.0035 mg/kg to about 1.4 mg/kg, or between about 0.0035 mg/kg to about 1.45 mg/kg, per day.

In some alternative embodiments, administration is effected once per day, or twice per day, or three times per day or four times per day, five times per day, or more.

As a treatment, the total time period for administration of the at least two MOR modulators, synergistic combinations and/or any combined composition thereof in accordance with the invention may be in some embodiments, between one day to four weeks or more, specifically, for one day, or for two days, or for four days, or for five days, or for six days, or for seven days, or for less than 1 week, or for 1 week, or for 2 weeks, or for 3 weeks, or for 4 weeks, specifically for one month, or for 5 weeks, or for 6 weeks, or for 7 weeks, or for 8 weeks, or for 2 months, or for 9 weeks, or for 10 weeks, or for 11 weeks, or for 12 weeks or for 3 months, or for 4 months, or for 5 months or for 6 months, or more.

In some specific embodiments, the time period for administration of the synergistically combined MOR activating modulators of the invention, or any combined compositions or kits thereof in accordance with the invention, may last until the pathologic condition, specifically at least one of, suicidality, mental disorder, mental pain and physical pain is resolved, relived or addressed. In some embodiments, such treatment course may continue for four weeks or more. It should be however appreciated that the treatment regimen may comprise two or more treatments cycles, with intervals, of between about one week to about four weeks or more, between each of the cycles.

Still further, in some embodiments, it should be understood that there are numerous administration routes that may be used by the methods of the invention. In some embodiments, the administration is at least one of oral, intravenous (IV), transdermal, mucosal, nasal, pulmonary, buccal or sublingual administration, or any combinations thereof. Other administration modes are also applicable, for example, subcutaneous, rectal, or parenteral (including intramuscular, intraperitoneal (IP), and intradermal) administration.

It should be appreciated that the at least two MOR modulators may be administered by the methods of the invention simultaneously. Alternatively, said at least two MOR modulators used by the invention, may be administered sequentially in either order.

In some embodiments, the therapeutic methods of the invention may be specifically applicable for treating, preventing, ameliorating and reducing acute suicidality in a subject in need.

As used herein, the phrase “acute suicidality” refers to a state wherein a subject (e.g., a person) is judged (e.g., by a practitioner such as a psychiatrist) to exhibit an acute risk of suicidality, i.e., a relatively severe risk for suicide in the near future, for example, within a period of 4 weeks. As used herein and in the art, the term “acute” refers to a condition with a relatively short, severe course. The onset of an acute risk is commonly associated with changes in a subject's circumstances and/or in some non-limiting embodiments, mental state. The acute suicidality may be identified as a condition in its own right or as a symptom of an underlying disorder. Thus, subjects having acute suicidality may suffer only from acute suicidality, or may suffer from a disorder known to be associated with acute suicidality or a disorder which is not associated with acute suicidality, as further discussed in detail hereinafter.

In comparison with an acute risk of suicide, a chronic risk of suicide is a longer-lasting risk, but a less severe risk at any given time. Chronic risk of suicide is commonly associated with a chronic mental illness (e.g., borderline personality disorder, chronic major depressive disorder, or chronic dysthymic disorder) and/or social and demographic factors.

It is to be appreciated that a subject may exhibit both an acute risk for suicide (a severe risk for the near future) and a chronic risk for suicide (a milder, but long-term risk), and the phrase “acute suicidality” is intended to encompass such cases.

In some embodiments of any of the aspects of the invention described herein, determining a presence of acute suicidality in a subject may be achieved using a test for measuring suicidality according to any technique used in the art.

In some embodiments, the test comprises a questionnaire filled out by a subject. Examples of tests for measuring suicidality include, without limitation, a Beck Suicidal Ideation (BSI) scale (as described in Beck and Steer [Manual for the Beck Scale or Suicide Ideation. San Antonio, Tex.: Psychological Corporation (1991)]); a Suicide Probability Scale (SPS) (as described in Cull and Gill [Suicide Probability Scale Manual. Los Angeles, Calif. (1988)]); a Columbia—Suicide Severity Rating Scale (C-SSRS) (as described in Posner et al. [CNS Spectr, 12: 156-162 (2007)]); and an Overt Aggression Scale Modified (OAS-M) (as described in Coccaro et al. [J Neuropsychiatry Clin Neurosci, 3:S44-51 (1991)]). Such tests comprise a questionnaire, wherein the answers to the questionnaire can be quantified to obtain value on a scale.

According to some embodiments of any of the aspects of the invention described herein, determining suicidality (e.g., as part of a method or treatment described herein) may comprise measuring suicidality on a BSI scale. For example, suicidality is optionally characterized by a score of at least 4, at least 5, at least 6, at least 7, at least 8, at least 9, at least 10, at least 11, at least 12, at least 13, at least 14, or at least 15 on a BSI scale. For this example, a subject that scores at least 4, at least 5, at least 6, at least 7, at least 8, at least 9, at least 10, at least 11, at least 12, at least 13, at least 14, or at least 15 on a BSI scale, is determined as a subject having acute suicidality. Therefore, in some embodiments, the synergistic MOR modulators of the invention may be administered to a subject diagnosed with suicidality, for any period required for recovery, as can be also classified using any of the tests described above.

In yet some further embodiments, the methods of the invention may be useful in treating, preventing, ameliorating, reducing or delaying the onset of at least one mental disorder. More specifically, such disorder may be at least one of mood disorder, psychotic disorder, personality disorder and anxiety disorder.

The term “mental disorder” as used herein refers generally to mild and severe mental illness health conditions, such as psychiatric or neuropsychiatric diseases, mood disorders, psychotic disorders, personality disorders, pre- and post-traumatic stress disorders, anxiety disorders, developmental disorders, learning disorders, sensory processing disorders, movement disorders, memory disorders, and behavioral disorders as well as other mental disorders and diseases. Such disorders are defined and categorized in the Diagnostic and Statistical Manual of Mental Disorders, 5^{th} Ed., Text Revision (“DSM-5”), American Psychiatric Association, Washington, D. C, 2013. The term “mental disorder”, as used herein, is not intended to imply a distinction between “physical” and “mental” disorders and is considered to encompass the full breadth of disorders described in DSM-5.

More specifically, in some specific embodiments, the methods of the invention may be applicable for at least one mood disorder, more specifically, at least one of mental pain, major depression, treatment resistant depression (TRD), mania, and bipolar disorders. In yet some further embodiments, the methods of the invention may be applicable for treating and preventing at least one psychotic disorder, for example, at least one of schizophrenia, schizoaffective disorder, schizophreniform disorder, delusional disorder, brief psychotic disorder, shared psychotic disorder, substance-induced psychotic disorder, psychotic disorder due to a medical condition and paraphrenia. Still further, in some embodiments, the methods of the invention may be particularly suitable for treating at least one personality disorder. Such disorder may be for example at least one of paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive disorder. In yet some further embodiments, the method of the invention may be applicable for at least one anxiety disorder such as at least one of panic disorder, GAD, specific phobia, SAD, separation anxiety disorder, agoraphobia, panic disorder, and selective mutism.

Thus, in some embodiments, the invention provide therapeutic and prophylactic methods applicable for at least one mood disorder. Herein, a “mood disorder” refers to a disorder where a disturbance in a person's mood is considered to be the main underlying feature, as determined by DSM-5 criteria. Mood disorders include major depression disorder (i.e., unipolar disorder), mania, dysphoria, bipolar disorder, dysthymia, cyclothymia and many others. See, e.g., Diagnostic and Statistical Manual of Mental Disorders, Fifth Edition, (DSM-5). Examples of mood disorders which may be associated with acute suicidality include, but are not limited to, a depressive disorder (including major depressive disorder, dysthymic disorder, and depressive disorder not otherwise specified) and a bipolar disorder (including bipolar disorder and cyclothymic disorder).

In yet some further embodiments, the invention provide therapeutic and prophylactic methods applicable for at least one personality disorder.

Herein, a “personality disorder” refers to an Axis II disorder according to DSM-5criteria, the disorder being associated with a person's personality type and/or behavior. Typically, a personality disorder may be associated with severe disturbances in behavior, which are generally associated with considerable personal and social disruption. Personality disorder may include at least one of paranoid personality disorder, Schizoid personality disorder, Schizotypal personality disorder, Antisocial personality disorder, Borderline personality disorder, Histrionic personality disorder, Narcissistic personality disorder, Avoidant personality disorder, Dependent personality disorder and Obsessive-compulsive personality disorder. More specifically, paranoid personality disorder may be characterized by a pattern of irrational suspicion and mistrust of others, interpreting motivations as malevolent. Schizoid personality disorder may be characterized by lack of interest and detachment from social relationships, apathy, and restricted emotional expression. Schizotypal personality disorder may be characterized by pattern of extreme discomfort interacting socially, and distorted cognitions and perceptions. Antisocial personality disorder may be characterized by pervasive pattern of disregard for and violation of the rights of others, lack of empathy, bloated self-image, manipulative and impulsive behavior. Borderline personality disorder may be characterized by pervasive pattern of abrupt mood swings, instability in relationships, self-image, identity, behavior and affect, often leading to self-harm and impulsivity. Histrionic personality disorder may be characterized by pervasive pattern of attention-seeking behavior and excessive emotions. Narcissistic personality disorder may be characterized by pervasive pattern of grandiosity, need for admiration, and a perceived or real lack of empathy. In a more severe expression, narcissistic personality disorder may show evidence of paranoia, aggression, psychopathy, and sadism, which is known as malignant narcissism. Avoidant personality disorder may be characterized by pervasive feelings of social inhibition and inadequacy, extreme sensitivity to negative evaluation. Dependent personality disorder may be characterized by pervasive psychological need to be cared for by other people. Obsessive-compulsive personality disorder may be characterized by characterized by rigid conformity to rules, perfectionism, and control to the point of satisfaction and exclusion of leisurely activities and friendships (distinct from obsessive-compulsive disorder). Further personality disorders may include sadistic personality disorder (pervasive pattern of cruel, demeaning, and aggressive behavior) and self-defeating personality disorder or masochistic personality disorder (characterized by behavior consequently undermining the person's pleasure and goals). Examples of personality disorders which may be associated with acute suicidality include, but are not limited to, a borderline personality disorder (characterized by unusual levels of instability in mood and black and white thinking), a narcissistic personality disorder (characterized by excessive preoccupation with issues of personal adequacy, power, prestige and vanity), and an antisocial personality disorder (characterized by a pervasive pattern of disregard for, and violation of, the rights of others).

In yet some further embodiments, the invention provide therapeutic and prophylactic methods applicable for at least one psychotic disorder. Herein, a “psychosis” refers to a mental state involving a gross deficit in reality testing. “Psychotic disorder”, as used herein, refers to a condition that affects the mind, resulting in at least some loss of contact with reality. Symptoms of a psychotic disorder include, e.g., hallucinations, changed behavior that is not based on reality, delusions and the like. See, e.g., DSM-5. Schizophrenia, schizoaffective disorder, schizophreniform disorder, delusional disorder, brief psychotic disorder, substance-induced psychotic disorder, and shared psychotic disorder are examples of psychotic disorders. Schizophrenia is a non-limiting example of psychosis which may be associated with acute suicidality. “Schizophrenia” refers to a psychotic disorder involving a withdrawal from reality by an individual. Symptoms comprise for at least a part of a month two or more of the following symptoms: delusions (only one symptom is required if a delusion is bizarre); hallucinations (only one symptom is required if hallucinations are of at least two voices talking to one another or of a voice that keeps up a running commentary on the patient's thoughts or actions); disorganized speech (e.g., frequent derailment or incoherence); grossly disorganized or catatonic behavior; or negative symptoms, i.e., affective flattening, alogia, or avolition. Schizophrenia encompasses disorders such as schizoaffective disorders. Diagnosis of schizophrenia is described in, e.g., DSM-5. Types of schizophrenia include, e.g., paranoid, disorganized, catatonic, undifferentiated, and residual.

In some further embodiments, the invention provide therapeutic and prophylactic methods applicable for substance abuse disorder. Herein, a “substance abuse disorder” encompasses both “substance abuse” and “substance dependence”, as these conditions are determined by DSM-5 criteria.

In yet some further embodiments, the invention provide therapeutic and prophylactic methods applicable for at least one anxiety disorder. Herein, an “anxiety disorder” refers to disorder associated with an abnormal and pathological fear and/or anxiety, as determined according to DSM-5 criteria. Examples of anxiety disorders which may be associated with acute suicidality include, but are not limited to, a social anxiety disorder (characterized by an intense fear of social situations, causing distress and impaired ability to function in at least some parts of daily life), a panic disorder (characterized by recurring severe panic attacks, e.g., experiencing sweat, chest pain, palpitations), a posttraumatic stress disorder (characterized by re-experiencing a traumatic event to which a subject has been exposed, for more than one month) and Specific phobias (an intense fear of a specific object or situation, such as heights or flying).

It should be further noted that the methods of the invention may be applicable for treating at least one of anorexia nervosa, a posttraumatic stress disorder, an adjustment disorder, an intermittent explosive disorder, an attention deficit disorder, a tic disorder, a body dysmorphic disorder, a dissociative identity disorder, a substance abuse disorder, a bipolar disorder, and a gender dysphoria.

More specifically, an “eating disorder”, as used herein, refers to a condition defined by abnormal eating habits, including either insufficient or excessive food intake, to the detriment of the individual's physical and/or mental health, as determined according to DSM-5 criteria. Anorexia nervosa is a non-limiting example of an eating disorder which may be associated with acute suicidality. Herein, an “attention deficit disorder” refers to the disorder “ADHD predominantly inattentive”, as defined by DSM-5 criteria, which is characterized by inattention, easy distractibility, disorganization, procrastination, forgetfulness and lethargy-fatigue. Herein, a “tic disorder” refers to a disorder characterized by tics (sudden, rapid, non-rhythmic, stereotyped, involuntary movements), as defined by DSM-5 criteria. Herein, a “gender dysphoria” refers to a disorder (also known in the art as “gender identity disorder”) defined, in accordance with DSM-5 criteria, by discontent of a person with his/her biological sex and/or the gender they were assigned at birth. Herein, a “dissociative disorder” refers to a condition that involves disruptions or breakdowns of memory, awareness, identity and/or perception, as determined according to DSM-5 criteria. A dissociative identity disorder, characterized by a person displaying multiple distinct identities or personalities (each with its own pattern of perceiving and/or interacting with the environment), is a non-limiting example of a dissociative disorder which may be associated with acute suicidality. Herein, a “somatoform disorder” refers to a mental disorder characterized, in accordance with DSM-5 criteria, by physical symptoms that suggest physical illness or injury, wherein the symptoms cannot be explained fully by a general medical condition, direct effect of a substance, or attributable to another mental disorder (e.g. panic disorder). Body dysmorphic disorder (characterized by excessive concern and preoccupation with a perceived defect in one's physical features) is a non-limiting example of a somatoform disorder which may be associated with acute suicidality. Herein, an “impulse control disorder” refers to a mental disorder characterized, according to DSM-5 criteria, by a failure to resist an impulsive act or behavior that may be harmful to self or others. Intermittent explosive disorder (characterized by an inability to control violent impulses) is a non-limiting example of an impulse control disorder which may be associated with acute suicidality. Herein, an “adjustment disorder” refers to a psychological response to an identifiable stressor or group of stressors that cause significant emotional or behavioral symptoms, as determined according to DSM-5 criteria. Still further, the invention provides compositions, kits, uses and methods that may be applicable for patients affected with TRD. Patients suffering from “treatment resistant depression” (TRD) include (1) those who fail to respond to standard doses (i.e., significantly superior to placebo in double-blind studies) of antidepressants (such as a monoamine oxidase inhibitors (MAOIs), tricyclic antidepressants (TCAs), tetracyclic antidepressants (TeCAs), selective serotonin reuptake inhibitors (SSRIs), and serotonin-norepinephrine reuptake inhibitors (SNRIs)) administered continuously for a minimum duration of 6 weeks, and (2) those who fail to respond to standard doses of an antidepressant (such as a monoamine oxidase inhibitors (MAOIs), tricyclic antidepressants (TCAs), tetracyclic antidepressants (TeCAs), selective serotonin reuptake inhibitors (SSRIs), and serotonin-norepinephrine reuptake inhibitors (SNRIs)) (monotherapy) administered continuously for a minimum duration of 12 weeks. One criteria for determining whether a patient's depression is treatment resistant to an antidepressant is if a Clinical Global Impression-Improvement (CGI-I) score of 1 (very much improved) or 2 (much improved) is not achieved by the end of a 6, 8, or 12 week trial. The CGI-I scale is defined in Guy, W. (ed.): ECDEU Assessment Manual for Psychopharmacology, Revised, DHEW Pub. No. (ADM) 76-338, Rockville, Md., National Institute of Mental Health, 1976.

In yet some further embodiments, the subject may be afflicted by a condition associated with an identifiable external event, for example, a psychological trauma. Herein, the phrase “psychological trauma” refers to an event which is, at least temporarily, psychologically stressful and/or life-threatening for a subject. It is emphasized that such an event does not necessarily have to satisfy criterion A of the DSM-5 definition of Posttraumatic Stress Disorder, or criterion A of the DSM-5 definition of Acute Stress Disorder.

In yet some further embodiments, the methods of the invention, as well as the combinations, combined compositions, kits and methods of the invention may be applicable for subjects afflicted with conditions associated with the use of a chemical substance, for example, a psychoactive substance. The acute suicidality may be associated either with a direct biological effect of the substance and/or with withdrawal symptoms caused when the subject ceases (permanently or temporarily) using the substance. Examples of substances which may be associated with acute suicidality include, but are not limited to, alcohol, amphetamines, opioids (e.g., heroin), cocaine (particularly during withdrawal), nicotine and benzodiazepines.

Alternatively, the subject may be afflicted by a medical condition or disorder which has no evident association with any identifiable external event and/or substance abuse. Such a condition or disorder may be due to genetic factors, environmental factors and/or random chance, although it may be difficult or impossible to determine which.

It should be appreciated that in some embodiments, the methods of the invention may be applicable for treating physical pain. As used herein, the term “physical pain” relates to acute or chronic unpleasant sensory and emotional experience associated with actual or potential tissue damage, or described in terms of such damage and includes the more or less localized sensation of discomfort, distress, or agony, resulting from the stimulation of specialized nerve endings. Pain can be categorized as nociceptive and neuropathic pain. Nociceptive can be visceral (felt in the internal organs of the body) or somatic (felt in the skin, and muscles). Visceral pain arises from inflammation or compression in or around internal organs. (pressure, aching or squeezing). Somatic pain can be either deep or superficial and arises when pain receptors in the tissues are stimulated. It is often localized pain and can be constant or stimulated by movement. Neuropathic or nerve pain is caused by damage or injury to various nerve fibers in the nervous system which leads to impaired pain processing. Nerve fibers become hypersensitive leading to lowering of the pain threshold, increasing pain sensitivity and even pain in the absence of stimuli.

There are many types of physiological pain applicable in the present invention, including, but not limited to neuropathic pain, inflammatory pain, nociceptive pain, orthopedic pain, specifically, pain of the musculoskeletal system, particularly the spine, joints, and muscles (e.g., variety of spinal dysfunctions including chronic back pain, scoliosis, and sacroiliac joint dysfunction), idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, chronic regional pain syndrome (CRPS), reflex sympathetic dystrophy, brachial plexus avulsion, neurogenic bladder, acute pain (e.g., musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, pain caused by at least one pathogenic agent, for example, a viral or bacterial pathogen, postherpetic pain, non-cardiac chest pain, irritable bowel syndrome and pain associated with bowel disorders and dyspepsia, pain associated with necrotic process, ulcers (e.g., diabetic ulcers or ulcers caused by vascular proliferative processes or injury), pain caused by any tearing, ripping, pulling and sheering of tissues or organs, pressure damage, deep vein thrombosis (DVT), pain caused by electrical shocks, pin, needles, stabbing or shooting and any combinations thereof.

It should be understood that the invention encompasses therapeutic methods, compositions, combinations and kits applicable for the treatment, prevention, amelioration and reduction of any physical pain at any aspect, duration, intensity, level or degree thereof. Still further, the invention encompasses therapeutic methods, compositions, combinations and kits applicable for any physical pain at any stage, degree or intensity as may be estimated, evaluated or classified by any method or scale known in the art, for example, at least one of Visual Analog Scale (VAS), Numeric Rating Scale (NRS) NRS and Mcgill Pain Questionnaire (MPQ). More specifically, in some embodiments, the therapeutic methods, compositions, combinations and kits may be applicable for a subject suffering from a physical pain at any degree or intensity as classified by VAS. The visual analogue scale or visual analog scale (VAS) is a psychometric response scale which can be used in questionnaires. It is a measurement instrument for subjective characteristics or attitudes that cannot be directly measured. When responding to a VAS item, respondents specify their level of agreement to a statement by indicating a position along a continuous line between two end-points. The pain VAS is a unidimensional measure of pain intensity, which has been widely used in diverse adult populations, including those with rheumatic diseases. The pain VAS is a continuous scale comprised of a horizontal (HVAS) or vertical (VVAS) line, usually 10 centimeters (100 mm) in length, anchored by 2 verbal descriptors, one for each symptom extreme. The pain VAS is a single-item scale. For pain intensity, the scale is most commonly anchored by “no pain” (score of 0) and “pain as bad as it could be” or “worst imaginable pain” (score of 100 [100-mm scale]). A higher score indicates greater pain intensity. Based on the distribution of pain VAS scores in postsurgical patients (knee replacement, hysterectomy, or laparoscopic myomectomy) who described their postoperative pain intensity as none, mild, moderate, or severe, the following cut points on the pain VAS have been recommended: no pain (0-4 mm), mild pain (5-44 mm), moderate pain (45-74 mm), and severe pain (75-100 mm). Normative values are not available.

In yet some further embodiments, the therapeutic methods, compositions, combinations and kits may be applicable for a subject suffering from a physical pain at any degree or intensity as classified by NRS. The NRS for pain is a unidimensional measure of pain intensity in adults, including those with chronic pain due to rheumatic diseases. Although various iterations exist, the most commonly used is the 11-item NRS. The NRS is a segmented numeric version of the visual analog scale (VAS) in which a respondent selects a whole number (0-10 integers) that best reflects the intensity of their pain. The common format is a horizontal bar or line. Similar to the pain VAS, the NRS is anchored by terms describing pain severity extremes. An 11-point numeric scale (NRS 11) with 0 representing one pain extreme (e.g., “no pain”) and 10 representing the other pain extreme (e.g., “pain as bad as you can imagine” and “worst pain imaginable”).

In some further embodiments, the therapeutic methods, compositions, combinations and kits may be applicable for a subject suffering from a physical pain at any degree or intensity as classified by MPQ. The Mcgill Pain Questionnaire (MPQ) is a multidimensional pain questionnaire designed to measure the sensory, affective and evaluative aspects of pain and pain intensity in adults with chronic pain, including pain due to rheumatic diseases. The scale contains 4 subscales evaluating the sensory, affective and evaluative, and miscellaneous aspects of pain, responses to which comprise the Pain Rating Index, and a 5-point pain intensity scale (Present Pain Intensity). The Pain Rating Index contains 78 pain descriptor items categorized into 20 subclasses, each containing 2-6 words that fall into 4 major subscales: sensory (subclasses 1-10), affective (subclasses 11-15), evaluative (subclass 16), and miscellaneous (subclasses 17-20). There is also a 1-item pain intensity scale. The value (score) associated with each descriptor is based on its position or rank order within the word set. The Present Pain Intensity scale, a measure of the magnitude of pain experienced by an individual, is a numeric-verbal combination that indicates overall pain intensity and includes 6 levels: none (0), mild (1), discomforting (2), distressing (3), horrible (4), and excruciating (5).

In yet some further specific embodiments, the methods of the invention may be specifically suitable for treating, preventing, ameliorating, reducing or delaying the onset of at least one of mental pain and depression. More specifically, the invention provide therapeutic and prophylactic methods applicable for mental pain. Mental pain relates to a wide range of psychological experiences characterized as a perception of negative changes in the self and its function that is accompanied by strong negative feelings. Intense unbearable mental (psychological) pain is defined as an emotionally based extremely aversive feeling which can be experienced as torment. It can be associated with a psychiatric disorder or with a severe emotional trauma. Terms such as mental pain, psychic pain, psychological pain, emptiness, psychache, internal perturbation, and psychological quality of life, are interchangeable, and may be used to refer to the same construct. The psychache is defined as an acute state of intense psychological pain associated with feelings of guilt, anguish, fear, panic, angst, loneliness and helplessness. The primary source of severe psychache ‘is frustrated psychological needs’. The psychache may be characterized by a method known in the art. Examples of suitable scales for measuring psychache include, for example, a Holden Psychache Scale (PAS) [Holden et al., Canadian J. Behay. Sci, 33:224-232 (2001)] and an Orbach and Mikulincer Mental Pain (OMMP scale) [Orbach et al., Suicide Life Threat Behav, 33:231-241 (2003)]. In some embodiments, a clinically significant level of psychache is characterized by a score of at least 24, optionally at least 27, optionally at least 30, optionally at least 33, optionally at least 36, optionally at least 39, optionally at least 42, optionally at least 45, optionally at least 48, optionally at least 51, and optionally at least 54, on the PAS scale. It should be understood that the invention is applicable for patients graded for any of the above scores of the PAS scale. In some additional embodiments, the invention provide therapeutic and prophylactic methods applicable for depression. The term “depression”, as used herein, refers to a mental state of depressed mood characterized by feelings of sadness, despair and discouragement. In some instances, depression is a clinical symptom, and can include, but not limited to, major depressive disorder (including single episode and recurrent), unipolar depression, treatment-refractory depression, resistant depression, anxious depression and dysthymia (also referred to as dysthymic disorder). Further, the term “depression” can encompass any major depressive disorder, dysthymic disorder, mood disorders due to medical conditions with depressive features, mood disorders due to medical conditions with major depressive-like episodes, substance-induced mood disorders with depressive features and depressive disorder not otherwise specific as defined by their diagnostic criteria, as listed in the American Psychiatric Association's Diagnostic and Statistical Manual of Mental Disorders, 5th Edition (DSM-5) or any later edition thereof, or the World Health Organization's International Statistical Classification of Diseases and Related Health Problems (ICD-10). More specifically, “Major depression disorder,” “major depressive disorder,” or “unipolar disorder” refers to a mood disorder involving any of the following symptoms: persistent sad, anxious, or “empty” mood; feelings of hopelessness or pessimism; feelings of guilt, worthlessness, or helplessness; loss of interest or pleasure in hobbies and activities that were once enjoyed, including sex; decreased energy, fatigue, being “slowed down”; difficulty concentrating, remembering, or making decisions; insomnia, early-morning awakening, or oversleeping; appetite and/or weight loss or overeating and weight gain; thoughts of death or suicide or suicide attempts; restlessness or irritability; or persistent physical symptoms that do not respond to treatment, such as headaches, digestive disorders, and chronic pain. Various subtypes of depression are described in, e.g., DSM-5, indicated above. In the DSM-5, depressive disorders are classified under mood disorders and include major depressive disorder, dysthymic disorder and depressive disorder not otherwise specified (or “atypical”). In general, regardless whether the depressive syndrome is melancholic, atypical, or some admixture of the two, a diagnosis of major depression is given when depressed mood is present, or loss of interest or pleasure in all activities is present, for at least two weeks. The term “major depressive disorder” (MDD) is understood in art, and refers to a diagnosis that is guided by diagnostic criteria listed in DSM-5 or ICD-10, or in similar nomenclatures (DSM-5, 5th Edition 2013). It should be appreciated that the compositions, kits, uses and methods of the invention may be applicable for any type of depression. Non-limiting examples for depressive conditions may include Atypical depression (AD), Atypical depression (AD) Melancholic depression, Psychotic major depression (PMD), Catatonic depression, Postpartum depression (PPD), Premenstrual dysphoric disorder (PMDD), Seasonal affective disorder (SAD), Dysthymia, Double depression, Depressive Disorder Not Otherwise Specified (DD-NOS), Depressive personality disorder (DPD), Recurrent brief depression (RBD), Minor depressive disorder. More specifically, Atypical depression (AD) is characterized by mood reactivity (paradoxical anhedonia) and positivity, significant weight gain or increased appetite (“comfort eating”), excessive sleep or somnolence (hypersomnia), a sensation of heaviness in limbs known as leaden paralysis, and significant social impairment as a consequence of hypersensitivity to perceived interpersonal rejection. Difficulties in measuring this subtype have led to questions of its validity and prevalence. Melancholic depression is characterized by a loss of pleasure (anhedonia) in most or all activities, a failure of reactivity to pleasurable stimuli, a quality of depressed mood more pronounced than that of grief or loss, a worsening of symptoms in the morning hours, early-morning waking, psychomotor retardation, excessive weight loss (not to be confused with anorexia nervosa), or excessive guilt. Psychotic major depression (PMD), or simply psychotic depression, is the term for a major depressive episode, in particular of melancholic nature, wherein the patient experiences psychotic symptoms such as delusions or, less commonly, hallucinations. These are most commonly mood-congruent (content coincident with depressive themes). Catatonic depression is a rare and severe form of major depression involving disturbances of motor behavior and other symptoms. Here, the person is mute and almost stuporose, and either is immobile or exhibits purposeless or even bizarre movements. Catatonic symptoms can also occur in schizophrenia or a manic episode, or can be due to neuroleptic malignant syndrome. Postpartum depression (PPD) is listed as a course specifier in DSM-5; it refers to the intense, sustained and sometimes disabling depression experienced by women after giving birth. Postpartum depression, which affects 10-15% of women, typically sets in within three months of labor, and lasts as long as three months. It is quite common for women to experience a short-term feeling of tiredness and sadness in the first few weeks after giving birth; however, postpartum depression is different because it can cause significant hardship and impaired functioning at home, work, or school as well as, possibly, difficulty in relationships with family members, spouses, or friends, or even problems bonding with the newborn. Women with personal or family histories of mood disorders are at particularly high risk of developing postpartum depression. Premenstrual dysphoric disorder (PMDD) is a severe and disabling form of premenstrual syndrome affecting 3-8% of menstruating women. The disorder consists of a “cluster of affective, behavioral and somatic symptoms” that recur monthly during the luteal phase of the menstrual cycle. The exact pathogenesis of the disorder is still unclear and is an active research topic. Treatment of PMDD relies largely on antidepressants that modulate serotonin levels in the brain via serotonin reuptake inhibitors as well as ovulation suppression using contraception. Seasonal affective disorder (SAD), also known as “winter depression” or “winter blues”, is a specifier. Some people have a seasonal pattern, with depressive episodes coming on in the autumn or winter, and resolving in spring. The diagnosis is made if at least two episodes have occurred in colder months with none at other times over a two-year period or longer. It is commonly hypothesized that people who live at higher latitudes tend to have less sunlight exposure in the winter and therefore experience higher rates of SAD, but the epidemiological support for this proposition is not strong (and latitude is not the only determinant of the amount of sunlight reaching the eyes in winter). It is said that this disorder can be treated by light therapy. SAD is also more prevalent in people who are younger and typically affects more females than males.

Dysthymia is a condition related to unipolar depression, where the same physical and cognitive problems are evident, but they are not as severe and tend to last longer (usually at least 2 years). The treatment of dysthymia is largely the same as for major depression, including antidepressant medications and psychotherapy. Double depression can be defined as a fairly depressed mood (dysthymia) that lasts for at least two years and is punctuated by periods of major depression. Depressive Disorder Not Otherwise Specified (DD-NOS) is designated by the code 311 for depressive disorders that are impairing but do not fit any of the officially specified diagnoses. According to the DSM-5, DD-NOS encompasses “any depressive disorder that does not meet the criteria for a specific disorder.” It includes the research diagnoses of recurrent brief depression, and minor depressive disorder listed below. Depressive personality disorder (DPD) is a controversial psychiatric diagnosis that denotes a personality disorder with depressive features. Originally included in the DSM-II, depressive personality disorder was removed from the DSM-III and DSM-III-R Recently, it has been reconsidered for reinstatement as a diagnosis. Depressive personality disorder was also described in Appendix B in the DSM-5-TR. In addition, depressive personality disorder is described in the last edition of the DSM-5 (2013). Recurrent brief depression (RBD), distinguished from major depressive disorder primarily by differences in duration. People with RBD have depressive episodes about once per month, with individual episodes lasting less than two weeks and typically less than 2-3 days. Diagnosis of RBD requires that the episodes occur over the span of at least one year and, in female patients, independently of the menstrual cycle. People with clinical depression can develop RBD, and vice versa, and both illnesses have similar risks. Minor depressive disorder, or simply minor depression, which refers to a mood disorder that does not meet the full criteria for major depressive disorder but at least two depressive symptoms are present for two weeks. These symptoms can be seen in many different psychiatric and mental disorders, which can lead to more specific diagnoses of an individual's condition. A person is considered to have minor depressive disorder if they experience 2 to 4 depressive symptoms, with one of them being either depressed mood or loss of interest or pleasure, during a 2-week period. The person must not have experienced the symptoms for 2 years and there must not have been one specific event that caused the symptoms to arise. In yet some further embodiments, the synergistic combinations, compositions, kits, uses and methods of the invention may be applicable for any subject diagnosed as having depression, specifically, any of the conditions and stages disclosed herein. In some specific embodiments, such subject may be diagnosed by any conventional test, for example, any of the tests disclosed herein. More specifically, any subject that has been defined as being afflicted with depression, as defined by any available test, specifically, at least one of the Montgomery-Åsberg Depression Rating Scale (MADRS), The Mini International Neuropsychiatric interview (M.I.N.I.) and Hamilton Depression Rating Scale. The Montgomery-Åsberg Depression Rating Scale (MADRS) is a test used by clinicians to assess the severity of depression among patients who have a diagnosis of depression. The MADRS includes 10 items and uses a 0 to 6 severity scale, scored following the interview. Higher scores indicate increasing depressive symptoms. Ratings can be added to form an overall score (range 0 to 50); no weights are used. Cut-off points include: 0 to 6—symptom absent, 7 to 19—mild depression, 30 to 34—moderate, 35 to 60—severe depression. The Mini International Neuropsychiatric interview (M.I.N.I.) is a tool to assist clinicians to conduct psychiatric diagnoses most often encountered, by using DSM criteria. This particular questionnaire was developed to evaluate the DSM-specifier “with depressive features” for (hypo-)manic episodes. It has been designed to be filled in directly by patients. A point is scored every time a patient answers yes to a question. In questions 2, 4 and 5, a point is scored if the patient answers yes to either a or b. If the total number of points is equal to or greater than 3, the patient presents a probable (hypo-) manic episode with depressive features. The Hamilton Depression Rating Scale (HDRS or also known as the Ham-D) is one of the most widely used clinician-administered depression assessment scale. The original version contains 17 items (HDRS17) pertaining to symptoms of depression experienced over the past week. A later 21-item version (HDRS21) included 4 items intended to subtype the depression. For the HDRS17, a score of 0-7 is generally accepted to be within the normal range (or in clinical remission), while a score of 20 or higher (indicating at least moderate severity) is usually required for entry into a clinical trial.

In some specific embodiments, the invention relates to a method of treating, preventing, ameliorating, reducing or delaying the onset of a physical pain in a subject in need thereof, comprising the step of administering to the subject a therapeutically effective amount of tianeptine and ketamine, any derivatives thereof or any pharmaceutically acceptable salts, esters or prodrugs thereof.

In some other specific embodiments, the invention relates to a method of treating, preventing, ameliorating, reducing or delaying the onset of suicidality in a subject in need thereof, comprising the step of administering to the subject a therapeutically effective amount of ketamine and buprenorphine, any derivatives thereof or any pharmaceutically acceptable salts, esters or prodrugs thereof.

In some other specific embodiments, the invention relates to a method of treating, preventing, ameliorating, reducing or delaying the onset of depression in a subject in need thereof, comprising the step of administering to the subject a therapeutically effective amount of ketamine and buprenorphine, any derivatives thereof or any pharmaceutically acceptable salts, esters or prodrugs thereof.

In yet another aspect, the invention provides a method of treating, preventing, ameliorating, reducing or delaying the onset of at least one of suicidality, a mental disorder and a physical pain, in subjects that are already treated with at least one MOR modulator. It should be appreciated that the subject may be pretreated with the MOR modulator, either for treating the same or related disorder or alternatively, for treating another or unrelated disorder. Thus, the methods provided herein may synergistically enhance the effect of the MOR modulator used for treating a certain subject. In yet some further embodiments, the methods provided herein extend the range of disorders treated in the same patient, specifically, provide a specific treatment regimen for treating at least two different disorders or conditions. More specifically, the therapeutic methods provided by this aspect of the invention may comprise the step of administering to a subject treated with at least one MOR modulator, a therapeutically effective amount of at least one additional different MOR modulator.

It should be noted that in some embodiments, the at least two MOR modulators used by the therapeutic methods of the invention, specifically, at least one MOR modulator that has been administered to a subject treated with a first MOR activator, synergistically activate MOR.

In some embodiments, the MOR modulators that may be used by the methods of the invention may be any one of buprenorphine, ketamine, tianeptine, dezocine, morphine, propoxyphene Oxycodone, Fentanyl, Heroin, Hydrocodone, Hydromorphone, Levorphanol, Meperidine, Methadone, Oxymorphone, Butorphanol, Pentazocine, Tramadol, Mitragynine and Codeine, any derivatives thereof or any pharmaceutically acceptable salts, esters, metabolite or prodrugs thereof, or any combinations, composition or kit comprising the same. It should be appreciated that any of the MOR modulators disclosed by the invention herein before in connection with other aspects of the invention, may be also applicable for this aspect as well. In some specific embodiments, the methods of the invention involve the step of administering to a subject treated with a first MOR modulator, an effective amount of a second modulator.

Thus, in some particular embodiments, the methods of the invention may comprise the step of administering to a subject treated with buprenorphine, an effective amount of Ketamine, any derivatives thereof or any pharmaceutically acceptable salts, esters, metabolite or prodrugs thereof. In further particular embodiments, the methods of the invention may comprise the step of administering to a subject treated with buprenorphine, an effective amount of tianeptine, any derivatives thereof or any pharmaceutically acceptable salts, esters, metabolite or prodrugs thereof. In some further embodiments, the methods of the invention may comprise the step of administering to a subject treated with tianeptine, an effective amount of Ketamine, any derivatives thereof or any pharmaceutically acceptable salts, esters, metabolite or prodrugs thereof.

In some further embodiments, the methods of the invention may comprise the step of administering to a subject treated with tianeptine, an effective amount of buprenorphine, any derivatives thereof or any pharmaceutically acceptable salts, esters, metabolite or prodrugs thereof.

In some further embodiments, the methods of the invention may comprise the step of administering to a subject treated with Ketamine, an effective amount of tianeptine, any derivatives thereof or any pharmaceutically acceptable salts, esters, metabolite or prodrugs thereof.

In some further embodiments, the methods of the invention may comprise the step of administering to a subject treated with Ketamine, an effective amount of buprenorphine, any derivatives thereof or any pharmaceutically acceptable salts, esters, metabolite or prodrugs thereof.

In yet some further specific embodiments, the methods of the invention involves the step of administering to a subject treated with a first MOR modulator, an effective amount of a second and a third MOR modulators. In some alternative embodiments, the methods of the invention involves the step of administering to a subject treated with a first MOR modulator and with a second MOR modulator, an effective amount of a third modulator.

In certain embodiments, the methods of the invention may comprise the step of administering to a subject treated with buprenorphine, an effective amount of tianeptine and ketamine, any derivatives thereof or any pharmaceutically acceptable salts, esters, metabolite or prodrugs thereof. In yet some further embodiments, the methods of the invention may comprise the step of administering to a subject treated with ketamine, an effective amount of tianeptine and buprenorphine, any derivatives thereof or any pharmaceutically acceptable salts, esters, metabolite or prodrugs thereof. In some further embodiments, the methods of the invention may comprise the step of administering to a subject treated with tianeptine, an effective amount of ketamine and buprenorphine, any derivatives thereof or any pharmaceutically acceptable salts, esters, metabolite or prodrugs thereof.

Still further, in some embodiments, the methods of the invention may comprise the step of administering to a subject treated with tianeptine and ketamine, an effective amount of buprenorphine, any derivatives thereof or any pharmaceutically acceptable salts, esters, metabolite or prodrugs thereof.

In some further embodiments, the methods of the invention may comprise the step of administering to a subject treated with tianeptine and buprenorphine, an effective amount of ketamine, any derivatives thereof or any pharmaceutically acceptable salts, esters, metabolite or prodrugs thereof. In some further embodiments, the methods of the invention may comprise the step of administering to a subject treated with ketamine and buprenorphine, an effective amount of tianeptine, any derivatives thereof or any pharmaceutically acceptable salts, esters, metabolite or prodrugs thereof. For enhancing the synergistic effect of the combined therapy provided by the invention, in some embodiments, buprenorphine may be administered to the subject in an amount ranging between about 0.000001 mg/kg to about 0.1 mg/kg, per day or 0.000001 mg/kg to about 0.01 mg/kg, per day, specifically, between about 0.00001 mg/kg to about 0.001 mg/kg, per day. In yet some further embodiments ketamine may be administered by the methods of the invention in an amount ranging between about 0.001 mg/kg to about 1 mg/kg, per day, specifically, in an amount ranging between about 0.005 mg/kg to about 0.5 mg/kg, per day. In some further embodiments, tianeptine may be administered by the methods of the invention in an amount ranging between about 0.001 mg/kg to about 1.5 mg/kg, per day specifically, in an amount ranging between about 0.0035 mg/kg to about 0.35 mg/kg, per day.

It should be noted that in some embodiments, the therapeutically effective amount of the combination may be administered for a total time period that ranges from one day to four weeks or more. In yet some further embodiments, the therapeutically effective amount of the combination of the invention may be administered once a day. In yet some further embodiments, the combined modulators of the invention may be administered twice a day or more, specifically, three, four, five, six, seven, eight, nine, ten times a day, or more. It should be noted that any of the treatment regimens disclosed by the invention in connection with other aspects of the invention, is also applicable for this aspect as well.

It should be noted that in some embodiments, the methods provided by this aspect may be applicable for treating, preventing, ameliorating and reducing acute suicidality in a subject in need. In some embodiments, the subject is already treated by at least one MOR modulator, and the method provided herein enhance the therapeutic effect of such treatment.

In some other embodiments, the methods of the invention are applicable in treating, preventing, ameliorating, reducing or delaying the onset of at least one mental disorder, such as, at least one mood disorder, psychotic disorder, personality disorder and anxiety disorder. In some embodiments, the subject may be already treated by at least one MOR modulator, and the method provided herein synergistically enhance the therapeutic effect of such treatment, or alternatively, extends the treatment for other conditions.

More specifically, in some specific embodiments, the methods of the invention may be applicable for at least one mood disorder, more specifically, at least one of mental pain, major depression, treatment resistant depression (TRD), mania, and bipolar disorders. In yet some further embodiments, the methods of the invention may be applicable for treating and preventing at least one psychotic disorder, for example, at least one of schizophrenia, schizoaffective disorder, schizophreniform disorder, delusional disorder, brief psychotic disorder, shared psychotic disorder, substance-induced psychotic disorder, psychotic disorder due to a medical condition and paraphrenia. Still further, in some embodiments, the methods of the invention may be particularly suitable for treating at least one personality disorder. Such disorder may be for example at least one of paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive disorder. In yet some further embodiments, the method of the invention may be applicable for at least one anxiety disorder such as at least one of panic disorder, GAD, specific phobia, SAD, separation anxiety disorder, agoraphobia, panic disorder, and selective mutism.

In some embodiments, the method of the invention may be applicable for treating, preventing, ameliorating, reducing or delaying the onset of at least one of mental pain and depression.

In yet some further embodiments, the methods of the invention may be applicable for treating pain, specifically, physical pain. More specifically, the methods of the invention may be applicable when a subject suffering from a pain is treated with at least one MOR modulator, specifically, activator. The administration of at least one additional MOR activator to the treated subject, may either enhance the effect on the pan, or alternatively, or additionally may further address additional condition in the treated subject, for example, suicidality, a mental disorder or even a physical pain of other source or type.

The invention provides therapeutic methods for treating variety of pathologic conditions, specifically, suicidality, mental disorders and physical pain.

It should be understood that the synergistic combinations, combined compositions, kits, uses and methods of the invention provide enhanced, increased, elevated, enlarged, augmented therapeutic effect that may be reflected by at least one of, achieving a desired therapeutic effect/outcome using reduced dosage of at least one of the MOR modulators, lowering frequency of administration, extending the length of intervals between two administrations and/or between cycles of administrations, reducing the number of administrations in each cycle and reducing the number of cycles required.

As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The terms “effective amount” or “sufficient amount” mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician). The terms “treat, treating, treatment” as used herein and in the claims mean ameliorating one or more clinical indicia of disease activity by administering a pharmaceutical composition of the invention in a patient having a pathologic disorder. The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above. The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing-additional symptoms and ameliorating or preventing the underlying metabolic causes of symptoms. The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state. The term “inhibit” and all variations of this term is intended to encompass the restriction or prohibition of the progress and exacerbation of pathologic symptoms or a pathologic process progress, said pathologic process symptoms or process are associated with. The term “eliminate” relates to the substantial eradication or removal of the pathologic symptoms and possibly pathologic etiology, optionally, according to the methods of the invention described below. The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

More specifically, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. The present invention relates to the treatment of subjects, or patients, in need thereof. By “patient” or “subject in need” it is meant any organism to whom the preventive and prophylactic combinations, composition/s, kit/s, and methods herein described is desired, including humans and domestic mammals. In some specific embodiments, the treated subject may be a human subject. The subject may be male or female, a child or an adult. In exemplary embodiments, the subject is an adult (e.g., at least 18 years old). The present invention relates to the treatment of subjects, or patients, in need thereof. It should be further noted that particularly in case of human subject, administering of the compositions of the invention to the patient includes both self-administration and administration to the patient by another person. Another aspect of the invention pharmaceutical composition for use in a method of treating, preventing, ameliorating, reducing or delaying the onset of at least one of at least one of suicidality, a mental disorder and a physical pain, in a subject in need thereof, the composition used by the invention may comprise as an active ingredient a therapeutically effective amount of a combination of at least two MOR modulators and optionally at last one pharmaceutically acceptable carrier. In some embodiments, the composition for use may be any of the combined compositions of the invention as described herein. In some embodiments, the invention provides compositions being packaged in a packaging material and identified in print, in or on said packaging material, for use in the treatment of acute suicidality in a subject in need thereof. More specifically, in some embodiments, the at least two MOR modulators comprised within the combined composition for use of the invention may synergistically activate MOR. In more specific embodiments, MOR modulators, specifically, MOR activating modulators may comprise at least one of Buprenorphine, Ketamine, Tianeptine, Dezocine, morphine, propoxyphene, Fentanyl, Heroin, Hydrocodone, Hydromorphone, Levorphanol, Meperidine, Methadone, Oxymorphone, Butorphanol, Pentazocine, Tramadol, Codeine, Mitragynine and Oxycodone, any derivatives thereof, or any pharmaceutically acceptable salts, esters, metabolites or prodrugs thereof.

Thus, in some specific embodiments, the combined composition for use of the invention may comprise Buprenorphine and Ketamine, any derivatives thereof or any pharmaceutically acceptable salts, esters, metabolites or prodrugs thereof.

In yet some further embodiments, the combined compositions for use of the invention may comprise Buprenorphine and Tianeptine, any derivatives thereof or any pharmaceutically acceptable salts, esters, metabolites or prodrugs thereof.

In some further embodiments, the combined compositions for use of the invention may comprise ketamine and tianeptine, any derivatives thereof or any pharmaceutically acceptable salts, esters, metabolites or prodrugs thereof.

In some alternative embodiments, the combined compositions for use of the invention may comprise buprenorphine, ketamine and tianeptine, any derivatives thereof or any pharmaceutically acceptable salts, esters, metabolite or prodrugs thereof.

In some specific and non-limiting embodiments, Buprenorphine may be present in an amount ranging between about 0.0001 mg to about 10 mg or specifically 0.0001 mg to about 1 mg, specifically, between about 0.001 mg to about 0.2 mg in the combination. In yet some further embodiments, Ketamine may be present in an amount ranging between about 0.01 mg to about 50 mg, specifically, between about 0.5 to 50 mg, more specifically, ranging between about 0.5 mg to about 17 mg in the combined composition of the invention. Still further, Tianeptine may be present in an amount ranging between about 0.01 mg to about 100 mg, more specifically, between about 0.25 mg to about 25 mg in said combination. In some embodiments, the amounts indicated herein refer to each dose of the combination of the invention. It should be appreciated that the amount used in the combined composition for use for each modulator varies and depend on the administration mode used and the specific disorder treated. In some embodiments, the pharmaceutical compositions for use in accordance with the invention may be applicable for treating, preventing, ameliorating and reducing acute suicidality in a subject in need. In yet some further embodiments, the pharmaceutical compositions for use in accordance with the invention may be applicable for treating, preventing, ameliorating, reducing or delaying the onset of at least one mental disorder. More specifically, such disorder may be at least one of mood disorder, psychotic disorder, personality disorder and anxiety disorder.

In further embodiments, the pharmaceutical compositions for use in accordance with the invention may be applicable for mood disorder, specifically, at least one of mental pain, major depression, TRD, mania, and bipolar disorders; for psychotic disorder, such as schizophrenia, schizoaffective disorder, schizophreniform disorder, delusional disorder, brief psychotic disorder, shared psychotic disorder, substance-induced psychotic disorder, psychotic disorder due to a medical condition and paraphrenia, for personality disorder such as paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive disorder; and for anxiety disorder such panic disorder, GAD, specific phobia, SAD, separation anxiety disorder, agoraphobia, panic disorder, and selective mutism. In some specific embodiments, the pharmaceutical composition for use according to the invention may be applicable for treating, preventing, ameliorating, reducing or delaying the onset of at least one of mental pain and depression. In yet some further specific embodiments, the pharmaceutical composition for use according to the invention may be applicable for treating, preventing, ameliorating, reducing or delaying the onset of at least one of physical pain, specifically, as disclosed by the invention. In more specific embodiments, the invention provides a pharmaceutical composition for use in a method of treating, preventing, ameliorating, reducing or delaying the onset of a physical pain, in a subject in need thereof, the combined composition may comprise Tianeptine and Ketamine, any derivatives thereof or any pharmaceutically acceptable salts, esters, metabolites or prodrugs thereof.

In some other specific embodiments, the invention provides a pharmaceutical composition for use in a method of treating, preventing, ameliorating, reducing or delaying the onset of suicidality, in a subject in need thereof, the combined composition may comprise Ketamine and Buprenorphine, any derivatives thereof or any pharmaceutically acceptable salts, esters, metabolites or prodrugs thereof.

In some other specific embodiments, the invention provides a pharmaceutical composition for use in a method of treating, preventing, ameliorating, reducing or delaying the onset of depression, in a subject in need thereof, the combined composition may comprise Ketamine and Buprenorphine, any derivatives thereof or any pharmaceutically acceptable salts, esters, metabolites or prodrugs thereof.

In yet another aspect, the invention provides at least two MOR modulators, or any compositions comprising the same, specifically, the at least two MOR modulators of the invention, for use in a method of treating, preventing, ameliorating, reducing or delaying the onset of at least one of suicidality, a mental disorder and a physical pain in a subject in need thereof.

In some embodiments, the at least two MOR modulators used by the invention synergistically activate MOR. In some embodiments, such MOR modulators may comprise at least one of buprenorphine, ketamine, tianeptine, dezocine, morphine, propoxyphene, Fentanyl, Heroin, Hydrocodone, Hydromorphone, Levorphanol, Meperidine, Methadone, Oxymorphone, Butorphanol, Pentazocine, Tramadol, Codeine, Mitragynine and Oxycodone or any derivatives thereof.

In yet some further embodiments, the at least two MOR modulators according to the invention may be used in a method of treating, preventing, ameliorating, reducing acute suicidality in a subject in need.

In yet some further embodiments, the at least two MOR modulators according to the invention may be applicable for use in a method of treating, preventing, ameliorating, reducing or delaying the onset of at least one mental disorder. In some embodiments, such mental disorder may be at least one of mood disorder, psychotic disorder, personality disorder and anxiety disorder.

It should be noted that all modulators, as well as conditions described herein above in connection with other aspects of the invention, are also applicable for these aspects as well.

In some embodiments, the invention provides an effective amount of buprenorphine, any derivatives thereof, or any pharmaceutically acceptable salts, esters, metabolites or prodrugs thereof and of ketamine any derivatives thereof, or any pharmaceutically acceptable salts, esters, metabolites or prodrugs thereof, or any pharmaceutical compositions and kits thereof for use in treating, preventing, ameliorating, reducing at least one of acute suicidality and depression in a subject in need.

In yet some further embodiments, the invention provides a method of treating, preventing, ameliorating, reducing or delaying the onset of at least one of suicidality and depression. More specifically, the method comprising the step of administering to a subject in need, specifically a subject suffering from at least one of suicidality and/or depression, an effective amount of buprenorphine, any derivatives thereof, or any pharmaceutically acceptable salts, esters, metabolites or prodrugs thereof and of ketamine any derivatives thereof, or any pharmaceutically acceptable salts, esters, metabolites or prodrugs thereof.

Still further, in some embodiments, the invention provides methods of treating, preventing, ameliorating, reducing or delaying the onset of at least one of suicidality and depression. The method comprising the step of administering to a subject treated with buprenorphine or any derivatives thereof, or any pharmaceutically acceptable salts, esters, metabolites or prodrugs thereof an effective amount of ketamine or any derivatives thereof, or any pharmaceutically acceptable salts, esters, metabolites or prodrugs thereof. In yet some alternative embodiments, the method comprises the step of administering to a subject treated with ketamine or any derivatives thereof, or any pharmaceutically acceptable salts, esters, metabolites or prodrugs thereof an effective amount of buprenorphine or any derivatives thereof, or any pharmaceutically acceptable salts, esters, metabolites or prodrugs thereof.

In yet some further embodiments, the invention provides an effective amount of Tianeptine, any derivatives thereof, or any pharmaceutically acceptable salts, esters, metabolites or prodrugs thereof and of ketamine, any derivatives thereof, or any pharmaceutically acceptable salts, esters, metabolites or prodrugs thereof, or any pharmaceutical composition and kits thereof for use in treating, preventing, ameliorating, reducing physical pain in a subject in need.

The invention further provides a method of treating, preventing, ameliorating, reducing or delaying the onset of physical pain. In some embodiments, the method of the invention may comprise the step of administering to a subject in need an effective amount of Tianeptine, any derivatives thereof, or any pharmaceutically acceptable salts, esters, metabolites or prodrugs thereof and of ketamine any derivatives thereof, or any pharmaceutically acceptable salts, esters, metabolites or prodrugs thereof.

Still further, the invention provides a method of treating, preventing, ameliorating, reducing or delaying the onset of at least one of physical pain. The method comprising the step of administering to a subject treated with Tianeptine or any derivatives thereof, or any pharmaceutically acceptable salts, esters, metabolites or prodrugs thereof, an effective amount of ketamine or of any derivatives thereof, or any pharmaceutically acceptable salts, esters, metabolites or prodrugs thereof. Alternatively, the method of the invention may comprise the step of administering to a subject treated with ketamine or any derivatives thereof, or any pharmaceutically acceptable salts, esters, metabolites or prodrugs thereof, an effective amount of tianeptine or of any derivatives thereof, or any pharmaceutically acceptable salts, esters, metabolites or prodrugs thereof.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%. It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric. The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof. It must be noted that, as used in this specification and the appended claims, the singular forms “a”, “an” and “the” include plural referents unless the content clearly dictates otherwise.

### EXAMPLES

Reference is now made to the following examples, which together with the above descriptions illustrate some embodiments of the invention in a non-limiting fashion.

### Materials:

### Bupronorphine Hydrochloride: Sigma-Aldrich PHR1729 (Lot #LRAA7438).

Tianeptine Sodium salt hydrate: Sigma-Aldrich T1692 (Lot #033M4727V).

### Ketamine Hydrochloride: Vetoquinol, Lure, France.

Hydroxy-Nor-Ketamine (rac-(2r,6r)-2 amino-2-(2-chlorophenyl)-6-hydroxycyclohexan-1-one; trifluoroacetic acid, trans): Enamine (Kyiv, Ukraine) EN300-1660923 (Lot #2017-0187421).

### Experimental Procedures

Tracking cAMP Secondary Messenger Pathway in MOR Expressing Cells (DiscoverX)

A Chinese hamster ovary cell line stably expressing non-tagged h-OPRM1 (human MOR) that signal through cAMP was used. The Hit Hunter® cAMP assay (for further details, see below) monitors the activation of this GPCR via Gi secondary messenger signaling in a homogenous, non-imaging assay format using a technology developed by DiscoverX called Enzyme Fragment Complementation (EFC) with 3-galactosidase ((3-Gal) as the functional reporter. The enzyme is split into two complementary portions: EA for Enzyme Acceptor and ED for Enzyme Donor. ED is fused to cAMP and in the assay competes with cAMP generated by cells for binding to a cAMP-specific antibody. Active (3-Gal is formed by complementation of exogenous EA to any unbound ED cAMP. Active enzyme can then convert a chemiluminescent substrate, generating an output signal detectable on a standard microplate reader.

Assay Design: GPCR cAMP Modulation

### Cell Handling:

1. cAMP Hunter cell line expressing h-OPRM1 was expanded from freezer stocks according to standard procedures.
2. Cells were seeded in a total volume of 20 μL into white walled, 384-well microplates and incubated at 37° C. for the appropriate time prior to testing.
3. cAMP modulation was determined using the DiscoverX HitHunter cAMP XS+ assay (for further details, see below).

### Gi Agonist Format:

1. For agonist determination, cells were incubated with sample in the presence of EC80 forskolin to induce cAMP-generation and Gi agonist activity, inhibiting cAMP formation, by the test compound was assayed, as follows:
2. Media was aspirated from cells and replaced with 15 μL 2:1 HBSS/10 mM Hepes: cAMP XS+Ab reagent.
3. Intermediate dilution of sample stocks was performed to generate 4× sample in assay buffer containing 4× EC80 forskolin. An amount of 4.5 μL of 4× sample was added to cells and incubated at 37° C. or room temperature for 30 or 60 minutes. The vehicle used for all stock solutions is DMSO and its final concentration in the assay system was 1%.

### Signal Detection

1. After appropriate compound incubation, assay signal was generated through incubation with 20 μL cAMP XS+ED/CL lysis cocktail for one hour followed by incubation with 20 μL cAMP XS+EA reagent for three hours at room temperature.
2. Microplates were read following signal generation with a PerkinElmer Envision™ instrument for chemiluminescent signal detection.

### Data Analysis

1. Compound activity was analyzed using CBIS data analysis suite (ChemInnovation, CA).
2. For Gi agonist mode assays, percentage activity is calculated using the following formula: % Activity=100%×(1−(mean RLU of test sample−mean RLU of MAX control)/(mean RLU of vehicle control−mean RLU of MAX control)).

RLU=Luciferase assay results which corresponds to chemiluminescence light intensity of the generated signal.

### Clinical Symptom Evaluation:

Suicidality is evaluated using the following methods:

Beck Suicidal Ideation (BSI) scale [Beck and Steer, Manual for the Beck Scale for Suicide Ideation. San Antonio, Tex.: Psychological Corporation (1991)];

Suicide Probability Scale (SPS) [Cull and Gill, Suicide Probability Scale Manual. Los Angeles, Calif. (1988)];

Columbia—Suicide Severity Rating Scale (C-SSRS) [Posner et al., CNS Spectr, 12: 156-162 (2007)]; and

Overt Aggression Scale Modified (OAS-M) [Coccaro et al., J Neuropsychiatry Clin Neurosci, 3:S44-51 (1991)].

Psychache is evaluated using the following methods:

Holden Psychache Scale (PAS) [Holden et al., Canadian J Behav Sci, 33:224-232 (2000]; and

Orbach and Mikulincer Mental Pain (OMMP) scale [Orbach et al., Suicide Life Threat Behav, 33:231-241 (2003)]. In addition, levels of depression are evaluated using a Beck Depression Inventory, second version (BDI-II) [Beck et al., J Pers Assess, 67:588-597 (1996)], a Hamilton Depression Rating Scale [Hamilton, M (1980) Rating depressive patients. Journal of Clinical Psychiatry. 41: 21-24], a Montgomery—Asberg Depression Rating Scale (MADRS) [Montgomery S A, Asberg M (April 1979). “A new depression scale designed to be sensitive to change”. British Journal of Psychiatry. 134 (4): 382-89. doi:10.1192/bjp.134.4.382. PMID 444788]; hopelessness is evaluated using a Beck Hopelessness Scale (BHS) [Beck and Steer, Manual for the Beck Hopelessness Scale. San Antonio, Tex.: Psychological Corporation (1988)]; general clinical impressions are evaluated using a Clinical Global Impressions (CGI) scale [Guy et al., Clinical Global Impressions. In: ECDEU Assessment Manual or Psychopharmacology. National Institute of Mental Health: Rockville. 218-222 (1976)]; attachment is assessed as described by Mikulincer and Erev [Br J Soc Psychol, 30:273-291 (1991)] and Mikulincer et al. [J Pers Soc Psychol, 58:273-280 (1990)]; and environmental and psychosocial factors contributing to disorders are determined according to Axis IV of the DSM-5.

### Statistics:

Data are analyzed using suitable software (e.g., SPSS software (version 14)). Demographic data were analyzed using a Fisher's Exact Test or a t-test.

### Example 1

### Buprenorphine and Ketamine Combination

OPRM1, the μ-opioid receptor (MOR), is a GPCR with high affinity for enkephalins and beta-endorphin, but a low affinity for dynorphins. The prototypical OPRM1 agonist is morphine, the primary psychoactive alkaloid in opium. It is an inhibitory G-protein coupled receptor that activates the Gi alpha subunit, inhibiting adenylate cyclase activity, thereby lowering cAMP levels. In order to test synergy of various drug combinations, towards the human receptor (h-OPRM1), a high sensitivity and absolute selectivity assay system, developed by DiscoverX (Fremont, Calif., USA) was employed. The analytical power of this assay, described in detail below, enables unequivocal identification of agonistic or antagonistic activity of drugs towards h-OPRM1, at the biologically relevant cellular context. Cells stably transfected with h-OPRM1 and expressing cAMP readout system were incubated with various concentrations of Buprenorphine and Ketamine active metabolite (hydroxynorketamine=HNK) combinations and assayed for Gi agonistic activity (see Experimental procedures). Final drug concentrations as well as the obtained percentage of inhibition of cAMP formation are specified in FIG. 1 and Table 1. A positive control dose-response was also examined in parallel (see FIG. 6).

TABLE 1
Bup/HNK Gi Agonistic Interactions
Agonistic activity (% of maximal c-AMP inhibition)

| Bup. [symbol] | | 0.07 | 0.42 | 2.5 | 15 | 92 | 550 |
|---|---|---|---|---|---|---|---|
| [symbol] HNK | 0 | nM | nM | nM | nM | nM | nM |
| 0 | | 7.8 | 79.2 | 100.4 | 99.7 | 98.1 | 97.4 |
| 1.4 nM | 1.1 | 47 | 84.1 | 99.9 | 99.8 | 99.3 | 97.2 |
| 8.5 nM | −11 | 37.6 | 85 | 100.1 | 100 | 99.4 | 98.1 |
| 50 nM | 1.3 | 25.3 | 79.5 | 99.4 | 99.8 | 98.5 | 98 |
| 300 nM | 10.3 | 19.1 | 85.5 | 99.5 | 100.2 | 98.2 | 96.6 |
| 1,800 nM | −3.4 | 23.7 | 84.9 | 101.5 | 99.1 | 97.1 | 98.9 |
| 11,000 nM | −8.6 | 37.6 | 88.2 | 100.3 | 99.9 | 98.7 | 99.2 |

Bup = Buprenorphine; HNK = Hydroxy-Nor-Ketamine

As clearly shown by FIG. 1 and Table 1, while the ketamine active metabolite by itself (HNK) has no agonistic activity, it enhances buprenorphine agonistic activity already at low concentration of both (HNK 1.4 nm=1.1%; Bup 0.07 nM=7.8%; combo response: 47%. Therefore, an outstanding synergistic effect was demonstrated for the combination of Buprenorphine and Ketamine at these concentrations. Without wishing to be bound by theory, these data provide a novel explanation for the mode of action of ketamine metabolite(s) vis a vis the h-OPRM1 receptor: while it has no agonistic activity by itself, it augments the activity of genuine mu agonist(s) on this receptor. Different concentrations of the Buprenorphine/HNK combination were also tested as follows:

Buprenorphine concentrations: 0.00016 nM, 0.008 nM, 0.04 nM, 0.2 nM, 1 nM and 5 nM

Hydroxy-Nor-Ketamine concentrations: 0.016 nM, 0.08 nM, 0.4 nM, 2 nM, 10 nM and 50 nM.

The obtained percentage of MOR activation (i.e. inhibition of cAMP formation) are specified in FIG. 2 and Table 2.

TABLE 2
Bup/HNK Gi Agonistic Interactions
Agonistic activity (% of maximal c-AMP inhibition)

| | HNK | | | | | | |
|---|---|---|---|---|---|---|---|
| | 0 | 0.00016 | 0.008 | 0.04 | 0.2 | 1 | 5 |
| Bup | | nM | nM | nM | nM | nM | nM |
| 0 | 0 | −4.3 | −12.5 | 7.6 | 28.8 | 90.8 | 99.4 |
| 0.016 nM | 0 | 0.2 | −6.7 | 20.6 | 36.9 | 89.2 | 100.2 |
| 0.08 nM | 0 | 3.1 | 1.4 | 16.9 | 33.7 | 81 | 96.5 |
| 0.4 nM | 0 | 7.6 | 19.4 | 18.2 | 44.7 | 89.2 | 100.2 |
| 2 nM | 0 | 6.3 | 12.9 | 28 | 57.3 | 89.2 | 99.8 |
| 10 nM | 0 | 7.1 | 8.8 | 34.1 | 43.9 | 85.1 | 100.2 |
| 50 nM | 0 | 6.7 | 21.4 | 30 | 41 | 88 | 99.4 |

Bup = Buprenorphine; HNK = Hydroxy-Nor-Ketamine

As clearly shown by FIG. 2 and Table 2, while the ketamine active metabolite by itself (HNK) has no agonistic activity, it enhances buprenorphine agonistic activity already at very low concentrations of both (HNK 0.4 nm=0%; Bup 0.008 nM=0%; combo response: 19.4%. Therefore, an outstanding synergistic effect was demonstrated for the combination of Buprenorphine and Ketamine at these low concentrations.

### Example 2

### Buprenorphine and Tianeptine Combination

Cells stably transfected with h-OPRM1 and expressing a cAMP readout system were incubated with various concentrations of Buprenorphine and Tianeptine combinations and assayed for Gi agonistic activity (see Experimental procedures). Final drug concentrations as well as the obtained percentage of cCAMP inhibition are specified in FIG. 3 and Table 3.

TABLE 3
Bup/Tia Gi Agonistic Interactions
Agonistic activity (% of maximal c-AMP inhibition)

| [symbol] | | | | | | | |
|---|---|---|---|---|---|---|---|
| Bup. [symbol] | 0 | 16 nM | 80 nM | 400 nM | 2,000 nM | 10,000 nM | 50,000 nM |
| 0 | | 25.6 | 52.6 | 94 | 98.3 | 97.1 | 97.9 |
| 0.07 nM | 7.8 | 75.6 | 88 | 97.4 | 98.6 | 97.7 | 96.7 |
| 0.42 nM | 79.2 | 85.9 | 90.4 | 96.6 | 97.2 | 96.3 | 96 |
| 2.5 nM | 100.4 | 96.1 | 91.1 | 97.3 | 96.8 | 97 | 97.1 |
| 15 nM | 99.7 | 97.9 | 95.7 | 98.5 | 97 | 95.1 | 96.4 |
| 92 nM | 98.1 | 99.3 | 97.3 | 98.3 | 95.1 | 97.1 | 96 |
| 550 nM | 97.4 | 98.1 | 96.6 | 97.9 | 99.1 | 97 | 96 |

Bup = buprenorphine; Tia = tianeptine

The Gi agonistic activity of the buprenorphine (Bup) plus tianeptine (Tia) combinations demonstrate clear synergy with respect to h-OPRM1, as depicted in Table 3 and FIG. 3. While Bup by itself at 0.07 nM yielded 7.8% response and Tia at 16 nM yielded 25.6% response, their combined response was 75.6%. This level of activity can be obtained by ×5 amount of Bup and by more than ×5 amount of Tia. Thus, this result reflects outstanding synergy. Tianeptine and buprenorphine synergize in their Gi agonistic activity vis a vis the h-OPRM1 receptor. This result enables achieving a desired therapeutic effect using lower dose of buprenorphine. Different concentrations of the Buprenorphine/Tianeptine combination were also tested as follows:

Buprenorphine concentrations: 0.00016 nM, 0.008 nM, 0.04 nM, 0.2 nM, 1 nM and 5 nM

Tianeptine concentrations: 0.64 nM, 3.2 nM, 16 nM, 80 nM, 400 nM and 2,000 Nm.

Final drug concentrations as well as the obtained percentage of MOR activation (i.e. cCAMP inhibition) are specified in FIG. 4 and Table 4.

TABLE 4
Bup/Tia Gi Agonistic Interactions
Agonistic activity (% of maximal c-AMP inhibition)

| | Bup. | | | | | | |
|---|---|---|---|---|---|---|---|
| | | 0.64 | 3.2 | 16 | 80 | 400 | 2,000 |
| Tia. | 0 | nM | nM | nM | nM | nM | nM |
| 0 | 0 | −14 | −1.4 | 5.9 | 59.4 | 91.2 | 96.1 |
| 0.00016 nM | −4.3 | −7.6 | 11.6 | 16.9 | 57.8 | 94.5 | 96.5 |
| 0.008 nM | −12.1 | 1.8 | 12.4 | 23.1 | 60.6 | 94.5 | 96.1 |
| 0.04 nM | 7.6 | 9.6 | 15.7 | 18.2 | 65.5 | 94.1 | 98.2 |
| 0.2 nM | 28.8 | 20.2 | 42.7 | 42.2 | 63.5 | 93.3 | 95.3 |
| 1 nM | 90.8 | 92.9 | 85.5 | 86.3 | 91.6 | 96.5 | 94.9 |
| 5 nM | 99.4 | 99.8 | 99 | 101.8 | 100.2 | 99.8 | 100.2 |

The Gi agonistic activity of the buprenorphine (Bup) plus tianeptine (Tia) combinations demonstrate clear synergy with respect to h-OPRM1, as depicted in Table 4 and FIG. 4. While Bup by itself at 0.008 nM nor Tia at 3.2 nM did not yield to any response, their combo response was 12.4%. Thus, this result reflects outstanding synergy. This result enables achieving a desired therapeutic effect using lower dose of buprenorphine.

### Example 3

### Ketamine Active Metabolite and Tianeptine Combination

Cells stably transfected with h-OPRM1 and expressing cAMP readout system were incubated with various concentrations of Ketamine active metabolite (hydroxynorketamine=HNK) and Tianeptine combination and assayed for Gi agonistic activity (see Experimental procedures). Final drug concentrations as well as the obtained percentage of cCAMP inhibition are specified in FIG. 5 and Table 5.

TABLE 5
Tia/HNK Gi Agonistic Interactions
Agonistic activity (% of maximal c-AMP inhibition)

| | HNK | | | | | | |
|---|---|---|---|---|---|---|---|
| | | 0.64 | 3.2 | 16 | 80 | 400 | 2,000 |
| Tia | 0 | nM | nM | nM | nM | nM | nM |
| 0 | −4.3 | −14 | −1.4 | 5.9 | 59.4 | 91.2 | 96.1 |
| 0.016 nM | 0.2 | 1.6 | 16 | 19.7 | 60.1 | 95.7 | 93.6 |
| 0.08 nM | 3.1 | 8.5 | 17 | 15.4 | 68.8 | 97.9 | 98.4 |
| 0.4 nM | 7.6 | 3.2 | 20.7 | 27.7 | 60.6 | 97.9 | 96.8 |
| 2 nM | 6.3 | 11.7 | 31.4 | 29.8 | 56.9 | 96.3 | 97.9 |
| 10 nM | 7.1 | 28.2 | 41.5 | 39.4 | 66 | 96.3 | 96.8 |
| 50 nM | 6.7 | 23.4 | 13.3 | 29.3 | 71.8 | 97.9 | 98.4 |

Tia = Tianeptine; HNK = Hydroxy-Nor-Ketamine

While HNK by itself at 0.4 nM yielded 7.6% response and Tia at 3.2 nM did not yield a response, their combo response was 20.7%. Moreover, when Tia at this non-active low concentration of 3.2 nM was combined with HNK 2 nM that by itself yielded threshold activity of 6.3%, together the response mounted to 31.4%; likewise, low Tia of 3.2 nM (no response) when combined with HNK 10 nM (7.1% response by itself) resulted in 41.5% level of activation (Table 5). Thus, this result reflects outstanding synergy.

### Example 4

### Effect of Combined Treatment of Buprenorphine/Ketamine, or Buprenorphine/Tianeptine, or Ketamine/Tianeptine Treatment on Acute Suicidality

A placebo-controlled double-blind study is designed in order to evaluate the effect of the combined treatments on acute suicidality. Patients suffering from suicidal ideation or behavior are recruited from primary and psychiatric care clinical settings across Israel. Patients include men and women, ages 18-60, who exhibited suicidal ideation or behavior, characterized by a score of more than six on the Beck Suicidal Ideation scale. Exclusion criteria are as follows: electroconvulsive therapy (ECT) within the last month; schizophrenia; current psychosis; substance abuse or alcohol abuse within the last two years; benzodiazepine dependence within the last 6 months; any significant systemic illness or unstable medical condition which does not permit inclusion, according to the research physician; pregnant and nursing women; and patients who currently suffer from severe impairment or dysfunction of liver, kidney, adrenal gland, gall, closed brain injury, urinary retention or respiratory system. Patients were randomly assigned buprenorphine treatment (7 patients) or placebo treatment (8 patients). Combined treatments and placebos are administered for four weeks. Patients receiving either combined treatments receive a final dose of 0.2 mg buprenorphine per day and 25 mg Tianeptine per day, or 0.2 mg buprenorphine per day and 35 mg Ketamine per day, or 25 mg tianeptine and 35 mg ketamine per day. All patients are evaluated by the Structured Clinical Interview for DSM-5, Axis I and II (SCID) [First et al., Biometrics Research Department, New York State Psychiatric Institute, New York (1995); First et al., American Psychiatric Press, Washington, D.C. (1997)]. Patients also fill up a demographic questionnaire which includes basic demographic information such as age, gender, education, income, etc., as well as information regarding clinical history. Each patient is interviewed 4 times, at weekly intervals, and the results are evaluated to determine clinical symptoms. Suicidality is evaluated according to a Beck Suicidal Ideation (BSI) scale, psychache is evaluated according to a Holden Psychache scale (PAS), and depression is evaluated according to a Beck Depression Inventory (BDI-II). The BSI scale is a scale of 0-38 (high score indicates a high degree of suicidality), based on 19 items in a questionnaire, wherein each item may be scored as 0, 1 or 2. The BSI questionnaire includes items for evaluating the active will to commit suicide (“active aspect”), will to not be alive (“passive aspect”), and potential lethality and the degree of concreteness of specific plans to commit suicide (“specific aspect”). These aspects are also scored separately. The PAS is a scale of 13-65 (high score indicates a high degree of psychache), based on 13 items in a questionnaire, wherein each item may be scored on a scale of 1-5. The BDI scale is a scale of 0-63 (scores of 14-19 indicate mild depression, 20-28 indicates moderate depression, and 29 or more indicates severe depression), based on 21 items in a questionnaire, wherein each item is scored on a scale of 0-3. The BDI questionnaire includes items for evaluating the severity of depressed feelings (“affective aspect”), physical signs and symptoms associated with depression (“somatic aspect”) and depressive thoughts (“cognitive aspect”) [Buckley et al., J Subst Abuse Treat, 20: 197-204 (2001)]. These three aspects are also scored separately. In addition, patients are examined 4 times, at weekly intervals, to confirm that no significant adverse side effects or worsening of mental health occurred.

### Example 5

### Effect of Combined Treatment of Buprenorphine/Ketamine, or Buprenorphine/Tianeptine, or Ketamine/Tianeptine Treatment on Depression

A placebo-controlled double-blind study is designed in order to evaluate the effect of the combined treatments on depression. Patients suffering from depression are recruited from primary and psychiatric care clinical settings across Israel. Patients include men and women, ages 18-75, who exhibited major depressive disorder, as determined by the Mini International Neuropsychiatric interview (M.I.N.I.) and a score of 19 or greater on the Montgomery-Åsberg Depression Rating Scale (MADRS). A senior psychiatrist carries out all screening assessments. Exclusion criteria included psychotic disorder or psychotic symptoms, bipolar disorder, alcohol or substance misuse, unstable medical illness. Combined treatments and placebos are administered for three months. Patients receiving either combined treatments receive a final dose of 0.2 mg buprenorphine per day and 25 mg Tianeptine per day, or 0.2 mg buprenorphine per day and 35 mg Ketamine per day, or 25 mg tianeptine and 35 mg ketamine per day. All patients are assessed for study eligibility in a personal interview conducted by a senior psychiatrist, within 24 h prior to first drug administration. The primary outcome measure is change in depression severity, as determined by the MADRS scores. Secondary outcomes are achievement of clinical remission, defined as a MADRS score ≤10 points, 17 and response to treatment, defined as a reduction of more than 50% of the baseline depression score, both at day 90.

### Example 6

### Effect of Combined Treatment of Buprenorphine/Ketamine, or Buprenorphine/Tianeptine, or Ketamine/Tianeptine Treatment on Chronic Pain (Low Back Pain)

A placebo-controlled double-blind study is designed in order to evaluate the effect of the combined treatments on Chronic Low Back Pain. Patients suffering from Low Back Pain over one year, diagnosed as having a degenerative disc disease as seen on magnetic resonance imaging (MRI), meeting minimum disc grading criteria: at least a grade III disc degeneration, a hyperintense zone, or abnormal disc morphology are recruited. Patients include men and women, ages 18-75, who exhibited Chronic Low Back Pain as specified above. Patients with pain due to disorders not including a component of disc degeneration, or those with unknown causes of pain are excluded. Patients with psychotic disorder or psychotic symptoms, bipolar disorder, alcohol or substance misuse, or unstable medical illness are also excluded. Combined treatments and placebos are administered daily for three months. Patients receiving either combined treatments receive a final dose of 0.2 mg buprenorphine per day and 25 mg Tianeptine per day, or 0.2 mg buprenorphine per day and 35 mg Ketamine per day, or 25 mg tianeptine and 35 mg ketamine per day. Participants rated their average lower back pain over the past 24 hours using an 11-point scale (0=no pain to 10=worst possible pain) and recorded it in an electronic diary. The percent change in pain score from baseline is calculated using weekly averages for up to 12 weeks.

1-55. (canceled)

56. A combination of at least two μ-opioid receptor (MOR) modulators, a combined composition, or a kit comprising said at least two MOR modulators, wherein said MOR modulators comprise at least one of Tianeptine, Ketamine and Buprenorphine, any derivatives thereof, or any pharmaceutically acceptable salts, esters, metabolites or prodrugs thereof, said composition optionally further comprises at least one pharmaceutically acceptable carrier, diluent, excipient and/or additive, optionally, said at least two MOR modulators synergistically activate MOR.

57. The combination, composition or kit according to claim 56, comprising Ketamine and Tianeptine, any derivatives thereof or any pharmaceutically acceptable salts, esters, metabolites or prodrugs thereof.

58. The combination, composition or kit according to claim 56, comprising Buprenorphine and Tianeptine, any derivatives thereof or any pharmaceutically acceptable salts, esters, metabolites or prodrugs thereof.

59. The combination, composition or kit according to claim 56, comprising any one of:
(a) Buprenorphine and Ketamine, any derivatives thereof or any pharmaceutically acceptable salts, esters, metabolites or prodrugs thereof; or
(b) Tianeptine, Ketamine and Buprenorphine, any derivatives thereof or any pharmaceutically acceptable salts, esters, metabolite or prodrugs thereof.

60. The combination, composition or kit according to claim 56, wherein said Buprenorphine is present in an amount ranging between about 0.001 mg to about 1 mg in said combination, said Ketamine is present in an amount ranging between about 0.5 mg to about 50 mg in said combination, and said Tianeptine is present in an amount ranging between about 0.25 mg to about 25 mg in said combination.

61. The combination, composition or kit according to claim 56, wherein said kit comprises at least two MOR modulators, each MOR modulator is provided in a pharmaceutical dosage form; said kit comprises any one of:
(I) (a) an effective amount of Tianeptine, any derivatives thereof, or any pharmaceutically acceptable salts, esters, metabolites or prodrugs thereof, optionally in a first pharmaceutical dosage form; and (b) an effective amount of Ketamine, any derivatives thereof, or any pharmaceutically acceptable salts, esters, metabolites or prodrugs thereof, optionally in a second pharmaceutical dosage form;
(II) (a) an effective amount of Buprenorphine, any derivatives thereof, or any pharmaceutically acceptable salts, esters, metabolites or prodrugs thereof, optionally in a first pharmaceutical dosage form; and (b) an effective amount of Tianeptine, any derivatives thereof, or any pharmaceutically acceptable salts, esters, metabolites or prodrugs thereof, optionally in a second pharmaceutical dosage form;
(III) (a) an effective amount of Buprenorphine, any derivatives thereof, or any pharmaceutically acceptable salts, esters, metabolites or prodrugs thereof, optionally in a first pharmaceutical dosage form; and (b) an effective amount of Ketamine, any derivatives thereof, or any pharmaceutically acceptable salts, esters, metabolites or prodrugs thereof, optionally in a second pharmaceutical dosage form; or
(IV) (a) an effective amount of Buprenorphine, any derivatives thereof or any pharmaceutically acceptable salts, esters, metabolites or prodrugs thereof, optionally in a first pharmaceutical dosage form; (b) an effective amount of Tianeptine, any derivatives thereof or any pharmaceutically acceptable salts, esters, metabolites or prodrugs thereof, optionally in a second pharmaceutical dosage form; and (c) an effective amount of Ketamine any derivatives thereof or any pharmaceutically acceptable salts, esters, metabolites or prodrugs thereof, optionally in a third pharmaceutical dosage form;
said kit optionally further comprises container means for containing said dosage forms.

62. The combination, composition or kit according to claim 61, comprising:
(a) an effective amount of Tianeptine, any derivatives thereof or any pharmaceutically acceptable salts, esters, metabolites or prodrugs thereof, optionally in a first pharmaceutical dosage form; and
(b) an effective amount of Ketamine, any derivatives thereof or any pharmaceutically acceptable salts, esters, metabolites or prodrugs thereof, optionally in a second pharmaceutical dosage form; and optionally
(c) container means for containing said first and second dosage forms.

63. A method of treating, preventing, ameliorating, reducing or delaying the onset of at least one of a physical pain, a mental disorder and suicidality in a subject in need thereof, comprising the step of administering to said subject a therapeutically effective amount of at least two MOR modulators, any combinations thereof, any combined compositions thereof or any kit comprising the same, wherein said MOR modulators comprise at least one of Tianeptine, Ketamine and Buprenorphine, any derivatives thereof, or any pharmaceutically acceptable salts, esters, metabolites or prodrugs thereof, optionally, said at least two MOR modulators synergistically activate MOR.

64. The method according to claim 63, comprising the step of administering to said subject a therapeutically effective amount of Ketamine and Tianeptine, any derivatives thereof or any pharmaceutically acceptable salts, esters, metabolite or prodrugs thereof.

65. The method according to claim 63, comprising the step of administering to said subject a therapeutically effective amount of Buprenorphine and Tianeptine, any derivatives thereof or any pharmaceutically acceptable salts, esters, metabolite or prodrugs thereof.

66. The method according to claim 63, comprising the step of administering to said subject a therapeutically effective amount of any one of:
(a) Buprenorphine and Ketamine, any derivatives thereof or any pharmaceutically acceptable salts, esters or prodrugs thereof; or
(b) Buprenorphine, Ketamine and tianeptine, any derivatives thereof or any pharmaceutically acceptable salts, esters, metabolite or prodrugs thereof.

67. The method according to claim 63, wherein said buprenorphine is administered in an amount ranging between about 0.00001 mg/kg/day to about 0.1 mg/kg/day, said Ketamine is administered an amount ranging between about 0.005 mg/kg/day to about 0.5 mg/kg/day, and said tianeptine is administered in an amount ranging between about 0.0035 mg/kg/day to about 0.35 mg/kg/day.

68. The method according to claim 63, for treating, preventing, ameliorating, reducing or delaying the onset of physical pain.

69. The method according to claim 63, for treating, preventing, ameliorating, reducing or delaying the onset of physical pain, the method comprising the step of administering to a subject in need an effective amount of Tianeptine, any derivatives thereof, or any pharmaceutically acceptable salts, esters, metabolites or prodrugs thereof and of ketamine any derivatives thereof, or any pharmaceutically acceptable salts, esters, metabolites or prodrugs thereof.

70. The method according to claim 63, for treating, preventing, ameliorating and reducing acute suicidality in a subject in need.

71. The method according to claim 63, for treating, preventing, ameliorating, reducing or delaying the onset of at least one mental disorder, said disorder is at least one of mood disorder, psychotic disorder, personality disorder and anxiety disorder, optionally, wherein said mood disorder is at least one of mental pain, major depression, treatment resistant depression (TRD), mania, and bipolar disorders; said psychotic disorder is at least one of schizophrenia, schizoaffective disorder, schizophreniform disorder, delusional disorder, brief psychotic disorder, shared psychotic disorder, substance-induced psychotic disorder, psychotic disorder due to a medical condition and paraphrenia; said personality disorder is at least one of paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive disorder; and said anxiety disorder is at least one of panic disorder, GAD, specific phobia, SAD, separation anxiety disorder, agoraphobia, panic disorder, and selective mutism.

72. The method according to claim 63, for treating, preventing, ameliorating, reducing or delaying the onset of at least one of mental pain and depression.

73. A method of treating, preventing, ameliorating, reducing or delaying the onset of at least one of suicidality, a mental disorder and a physical pain, the method comprising the step of administering to a subject treated with at least one MOR modulator, a therapeutically effective amount of at least one additional MOR modulator, wherein said MOR modulators comprise at least one of Tianeptine, Ketamine and Buprenorphine, any derivatives thereof, or any pharmaceutically acceptable salts, esters, metabolites or prodrugs thereof, optionally, wherein said at least one MOR modulator and said at least one additional modulator synergistically activate MOR.

74. The method according to claim 73, wherein said method comprises the step of any one of:
(a) administering to a subject treated with Tianeptine, an effective amount of Ketamine, any derivatives thereof or any pharmaceutically acceptable salts, esters, metabolite or prodrugs thereof;
(b) administering to a subject treated with Tianeptine, an effective amount of Buprenorphine, any derivatives thereof or any pharmaceutically acceptable salts, esters, metabolite or prodrugs thereof;
(c) administering to a subject treated with Buprenorphine, an effective amount of Ketamine, any derivatives thereof or any pharmaceutically acceptable salts, esters, metabolite or prodrugs thereof; or
(d) administering to a subject treated with Buprenorphine, an effective amount of Tianeptine and Ketamine, any derivatives thereof or any pharmaceutically acceptable salts, esters, metabolite or prodrugs thereof.

75. The method according to claim 63, for treating, preventing, ameliorating, reducing or delaying the onset of at least one of physical pain, the method comprising the step of administering to a subject treated with Tianeptine or any derivatives thereof, or any pharmaceutically acceptable salts, esters, metabolites or prodrugs thereof, an effective amount of Ketamine or of any derivatives thereof, or any pharmaceutically acceptable salts, esters, metabolites or prodrugs thereof.


SEQUENCE LISTING (5 sequences)

SEQ ID NO: 1 - protein, 400 aa, Homo sapiens
  MISC_FEATURE: Homo sapiens MOR-1 as denoted by NCBI Reference Sequence NP_000905.3
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 2 - protein, 418 aa, Homo sapiens
  MISC_FEATURE: mu-type opioid receptor isoform MOR-1O NCBI Reference Sequence NP_001008503.2
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQPPLAVSMAQIFTRYPPPTHREKTCNDYMKR

SEQ ID NO: 3 - protein, 392 aa, Homo sapiens
  MISC_FEATURE: mu-type opioid receptor isoform MOR-1A NCBI Reference Sequence NP_001008504.2
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQVRSL

SEQ ID NO: 4 - protein, 446 aa, Homo sapiens
  MISC_FEATURE: mu-type opioid receptor isoform MOR-1X NCBI Reference Sequence NP_001008505.2
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQCLPIPSLSCWALEQGCLVVYPGPLQGPLVRYDLPAILHSSCLRGNTAPSPSGGAFLLS

SEQ ID NO: 5 - protein, 493 aa, Homo sapiens
  MISC_FEATURE: mu-type opioid receptor isoform MOR-1i  NCBI Reference Sequence NP_001138751.1
MCLHRRVPSEETYSLDRFAQNPPLFPPPSLPASESRMAHAPLLQRCGAARTGFCKKQQELWQRRKEAAEALGTRKVSVLLATSHSGARPAVSTMDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP